(19) United States
(12) Patent Application Publication
(10) Pub. No.: US 2016/0199479 A1
(43) Pub. Date: Jul. 14, 2016

(54) T cell receptors

(71) Applicant: IMMUNOCORE LIMITED, Abingdon (GB)

(72) Inventors: Qin SU, Abingdon (GB); Peter MOLLOY, Abingdon (GB); Nathaniel LIDDY, Abingdon (GB)

(21) Appl. No.: 15/017,765

(22) Filed: Feb. 8, 2016

Related U.S. Application Data
(63) Continuation-in-part of application No. PCT/GB2014/052464, filed on Aug. 12, 2014.

(30) Foreign Application Priority Data
Aug. 12, 2013 (GB) 1314404.3

(51) Int. Cl. A61K 39/12; C07K 16/28; C12N 7/00

(52) U.S. Cl. CPC A61K 39/12; C12N 7/00; C07K 16/2809; C07K 2319/30; C07K 2319/03; C07K 2317/622; C12N 2710/16271; C12N 2710/16234; C12N 2710/16222; A61K 2039/6056

(57) ABSTRACT

The present invention relates to T cell receptors (TCRs) which bind the HLA-A2 restricted CLGGLLTMV peptide (SEQ ID NO: 1) derived from the LMP2A protein from Epstein Barr Virus (EBY). TCRs of the invention comprise a TCR alpha chain variable domain and/or a TCR beta variable domain. Certain preferred TCRs also bind the natural peptide variants SLGGLLTMV (SEQ ID NO: 17) and CLGGLITMV (SEQ ID NO: 18) presented as a peptide-HLA-A2 complex. The TCRs of the invention demonstrate excellent specificity profiles for those LMP2A epitopes and have binding affinities for the complex which result in an enhanced ability to recognize the complex compared to a soluble reference TCR having the extracellular sequence of the native EBY LMP2A TCR alpha chain given in FIG. 3 (SEQ ID No: 4) and the extracellular sequence of the native EBY LMP2A TCR beta chain given in FIG. 4 (SEQ ID No: 5).

### RELATED APPLICATIONS AND INCORPORATION BY REFERENCE

This application is a continuation-in-part application of international patent application Serial No. PCT/GB2014/052464 filed 12 Aug. 2014, which published as PCT Publication No. WO 2015/022520 on 19 Feb. 2015, which claims benefit of GB patent application Serial No. 1314404.3 filed 12 Aug. 2013.

The foregoing applications, and all documents cited therein or during their prosecution (“appln cited documents”) and all documents cited or referenced in the appin cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

The instant application contains a Sequence Listing which has been submitted electronically in ASCII format and is hereby incorporated by reference in its entirety. Said ASCII copy, created on Mar. 28, 2016, is named 44172_00_2022_SL.txt and is 34,762 bytes in size.

### FIELD OF THE INVENTION

The present invention relates to T cell receptors (TCRs) which bind the HLA-A2 restricted CLGGLLTMV peptide (SEQ ID NO: 1) derived from the LMP2A protein from Epstein Barr Virus (EBV); the TCRs comprising alpha and/or beta variable domains that are mutated relative to the native LMP2A TCR alpha and/or beta variable domains. Certain preferred TCRs also bind the natural peptide variants SLGGLLTMV (SEQ ID NO: 17) and CLGGLITMV (SEQ ID NO: 18) presented as a peptide-HLA-A2 complex. The TCRs of the invention demonstrate excellent specificity profiles for those LMP2A epitopes and have binding affinities for the complex which result in an enhanced ability to recognize the complex compared to the reference EBV LMP2A TCR described below.

### BACKGROUND TO THE INVENTION

Epstein Barr Virus (EBV) is one of the most common human viruses worldwide, and most people become infected with EBV sometime during their lives. In the US as many as 95% of adults between 35 and 40 years of age have been infected. EBV during adolescence or young adulthood causes infectious mononucleosis in 35% to 50% of cases with symptoms including fever, sore throat and swollen lymph glands. Although the symptoms usually disappear within 1 or 2 months, EBV remains dormant or latent in a few cells in the throat and blood for the rest of the person's life. Periodically, the virus can reactivate but usually without symptoms of illness. EBV also establishes a lifelong dormant infection in some cells of the body's immune system. Many healthy people can carry and spread the virus intermittently for life. These people are usually the primary reservoir for person-to-person transmission.

EBV infection has been associated with a number of malignant and non-malignant diseases (Kutok, J. L. et al. Annu Rev Pathol 2006. 1: 375-404), including mononucleosis, Burkitt's lymphoma, nasopharyngeal carcinoma (NPC) and Hodgkin's lymphoma (HL). There is also evidence for EBV-associated autoimmunity.

LMP2A is one of the few EBV genes expressed in all type II and type III diseases/malignancies. LMP2A is a transmembrane protein that acts as a negative modulator of B cell-receptor signaling and promotes cell survival via the sequestering of tyrosine kinases. The HLA-A2 restricted peptide, CLGGLLTMV (SEQ ID No: 1), is the most immunodominant LMP epitope in latent disease, with epitope-specific cytotoxic T lymphocytes detectable in 60-75% of individuals ex vivo. The CLGGLLTMV (SEQ ID NO: 1) peptide has long been seen to be a potential target for NPC and HL treatments, since the epitope is conserved in biopsies taken from NPC and HL patients and, along with other EBV latent epitopes, is immunologically weak.

Therefore, the HLA-A2 restricted CLGGLLTMV peptide (SEQ ID NO: 1) (and including the natural variants SLGGLLTMV (SEQ ID No: 17) and CLGGLITMV (SEQ ID No: 18)), provides a suitable disease marker that the EBV TCRs of the invention can target. TCRs of the invention may be transformed into T-cells, rendering them capable of destroying EBV infected cells presenting that HLA complex, for administration to a patient in the treatment process known as adoptive therapy. For this purpose, it would be desirable if the TCRs had a higher affinity and/or a slower off-rate for the peptide-HLA complex than native TCRs specific for that complex. Dramatic increases in affinity have been associated with a loss of antigen specificity in TCR gene-modified CD8⁺ T cells, which could result in the nonspecific activation of these TCR-transfected CD8⁺ cells, so TCRs having a somewhat higher affinity and/or a slower off-rate for the peptide-HLA complex than native TCRs specific for that complex, but not a dramatically higher affinity and/or dramatically slower off-rate for the peptide-HLA complex than native TCRs, would be preferred for adoptive therapy (see Zhao et al., (2007) J Immunol. 179: 5845-54; Robbins et al., (2008) J Immunol. 180: 6116-31; and WO2008/038002). Some TCRs of the invention may be useful for the purpose of delivering cytotoxic or immune effector agents to the EBV infected cells. For this use it is desirable that the TCRs have a considerably higher affinity and/or a slower off-rate for the peptide-HLA complex than native TCRs specific for that complex. For example, the binding affinity may be at least double that of the reference EBV LMP2A TCR described below.

TCRs are described using the International Immunogenetics (IMGT) TCR nomenclature, and links to the IMGT public database of TCR sequences. Native alpha-beta heterodimeric TCRs have an alpha chain and a beta chain. Broadly, each chain comprises variable, joining and constant regions, and the beta chain also usually contains a short diversity region between the variable and joining regions, but this diversity region is often considered as part of the joining region. Each variable region comprises three CDRs (Complementarity Determining Regions) embedded in a framework sequence, one being the hypervariable region named CDR3. There are several types of alpha chain variable (Va) regions and several types of beta chain variable (Vβ) regions distinguished by their framework, CDR1 and CDR2 sequences, and by a partly defined CDR3 sequence. The Va types are referred to in IMGT nomenclature by a unique TRAV number. Thus “TRAV21” defines a TCR Vα region having unique framework and CDR1 and CDR2 sequences, and a CDR3 sequence which is partly defined by an amino acid sequence which is preserved from TCR to TCR but which also includes an amino acid sequence which varies from TCR to TCR. In the same way, “TRBV5-1” defines a TCR VP region having unique framework and CDR1 and CDR2 sequences, but with only a partly defined CDR3 sequence.

The joining regions of the TCR are similarly defined by the unique IMGT TRAJ and TRBJ nomenclature, and the constant regions by the IMGT TRAC and TRBC nomenclature.

The beta chain diversity region is referred to in IMGT nomenclature by the abbreviation TRBD, and, as mentioned, the concatenated TRBD/TRBJ regions are often considered together as the joining region.

The α and β chains of αβ TCR's are generally regarded as each having two “domains”, namely variable and constant domains. The variable domain consists of a concatenation of variable region and joining region. In the present specification and claims, the term “TCR alpha variable domain” therefore refers to the concatenation of TRAV and TRAJ regions, and the term TCR alpha constant domain refers to the extracellular TRAC region, or to a C-terminal truncated TRAC sequence. Likewise the term “TCR beta variable domain” refers to the concatenation of TRBV and TRBD/TRBJ regions, and the term TCR beta constant domain refers to the extracellular TRBC region, or to a C-terminal truncated TRBC sequence.

The unique sequences defined by the IMGT nomenclature are widely known and accessible to those working in the TCR field. For example, they can be found in the IMGT public database. The “T cell Receptor Factsbook”, (2001) LeFranc and LeFranc, Academic Press, ISBN 0-12-441352-8 also discloses sequences defined by the IMGT nomenclature, but because of its publication date and consequent time-lag, the information therein sometimes needs to be confirmed by reference to the IMGT database.

Citation or identification of any document in this application is not an admission that such document is available as prior art to the present invention.

### SUMMARY OF THE INVENTION

Applicants obtained a native EBV LMP2A TCR (Clone SB34, from John Miles, Queensland Institute of Medical Research, Brisbane, Australia). The DNA and corresponding amino acid sequence of this TCR is available to download from the following website http://www.tcells.org/scientific/johnmiles/ and has the following alpha and beta chain usage:
Alpha chain: TRAV12-3*01/TRAJ41*01/TRAC (the extracellular sequence of the native EBV LMP2A TCR alpha chain is given in FIG. 1 (SEQ ID No: 2). The CDRs are defined by amino acids 27-32 (CDR1), 50-54 (CDR2) and 90-102 (CDR3) of SEQ ID NO: 2.
Beta chain: TRBV11-2*01/TRBD1/TRBJ2-7*01/TRBC2 (the extracellular sequence of the native EBV LMP2A TCR alpha chain is given in FIG. 2 (SEQ ID No: 3). The CDRs are defined by amino acids 27-31 (CDR1), 49-54 (CDR2) and 94-102 (CDR3) of SEQ ID NO: 3.

(Note, the term ‘*01’ indicates there is more than one allelic variant for this sequence, as designated by IMGT nomenclature, and that it is the *01 variant which is present in the TCR clone referred to above. Note also that the absence of a “*” qualifier means that only one allele is known for the relevant sequence.)

The terms “wild type TCR”, “native TCR”, “wild type EBV LMP2A TCR”, and “native EBV LMP2A TCR” are used synonymously herein to refer to this naturally occurring TCR having the extracellular alpha and beta chain SEQ ID Nos: 2 and 3 respectively.

For the purpose of providing a reference TCR against which the binding profile of TCRs of the invention may be compared, it is convenient to use the soluble TCR having the extracellular sequence of the native EBV LMP2A TCR alpha chain given in FIG. 3 (SEQ ID No: 4) and the extracellular sequence of the native EBV LMP2A TCR beta chain given in FIG. 4 (SEQ ID No: 5). That TCR is referred to herein as the “the reference TCR” or “the reference EBV LMP2A TCR”. Note that SEQ ID No: 4 is identical to the native alpha chain extracellular sequence SEQ ID No: 2 except that C161 has been substituted for T161 (i.e. T48 of TRAC). Likewise SEQ ID No: 5 is identical to the native beta chain extracellular sequence SEQ ID No: 3 except that C169 has been substituted for S169 (i.e. S57 of TRBC2), A187 has been substituted for C187 and D201 has been substituted for N201. These cysteine substitutions relative to the native alpha and beta chain extracellular sequences enable the formation of an interchain disulfide bond which stabilises the refolded soluble TCR, ie the TCR formed by refolding extracellular alpha and beta chains. Use of the stable disulfide linked soluble TCR as the reference TCR enables more convenient assessment of binding affinity and binding half life.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 (SEQ ID No: 2) gives the amino acid sequence of the extracellular part of the alpha chain of a wild type EBV LMP2A-specific TCR with gene usage TRAV12-3*01/TRAJ41*01/TRAC.

FIG. 2 (SEQ ID No: 3) gives the amino acid sequence of the extracellular part of the beta chain of a wild type EBV LMP2A-specific TCR TRBV11-2*01/TRBD1/TRBJ2-7*01/TRBC2 beta chain amino acid sequence.

FIG. 3 (SEQ ID No: 4) gives the amino acid sequence of the alpha chain of a soluble TCR (referred to herein as the “reference TCR”). The sequence is the same as that of FIG. 1 except that a cysteine (bold and underlined) is substituted for T161 of SEQ ID No: 2 (i.e. T48 of the TRAC constant region).

FIG. 4 (SEQ ID No: 5) gives the amino acid sequence of the beta chain of a soluble TCR (referred to herein as the “reference TCR”). The sequence is the same as that of FIG. 2 except that a cysteine (bold and underlined) is substituted for S169 of SEQ ID No: 3 (i.e. S57 of the TRBC2 constant region) and A187 is substituted for C187 and D201 is substituted for N201.

FIG. 5 (SEQ ID Nos: 6-12) gives the amino acid sequence of the alpha chains which may be present in TCRs of the invention. The subsequences forming the CDR regions, or substantial parts of the CDR regions are underlined. Residues mutated relative to the native alpha chain (SEQ ID No 2) are shaded. An introduced cysteine referred to in relation to FIG. 3 is shown bold and underlined.

FIG. 6 (SEQ ID No: 13), gives the amino acid sequence of the beta chain which may be present in TCRs of the invention. The subsequences forming the CDR regions, or substantial parts of the CDR regions are underlined. Residues mutated relative to the native alpha chain (SEQ ID No 2) are shaded. An introduced cysteine (referred to in relation to FIG. 4) is shown bold and underlined and, also relative to the wild type sequence in FIG. 2, C187 has been mutated to A187 and D201 is substituted for N201 to eliminate an unpaired cysteine in any alpha-beta TCR having these beta chains.

FIG. 7 (SEQ ID No: 14) and (SEQ ID No: 15) give DNA sequences encoding the TCR alpha and beta chains respectively for FIGS. 3 and 4 (introduced cysteines are shown in bold).

FIG. 8 (SEQ ID NO: 16) gives the amino acid sequence of an anti-CD3 scFv antibody fragment (bold type) fused via a linker namely GGGGS (SEQ ID NO: 23) (underlined) at the N-terminus of an EBV LMP2A β chain. The EBV LMP2A TCR β chain is that of FIG. 6 (SEQ ID No: 13).

FIGS. 9A-B show the results of the assays described in Example 6 for the TCR-scFv antibody fusions prepared according to Example 5.

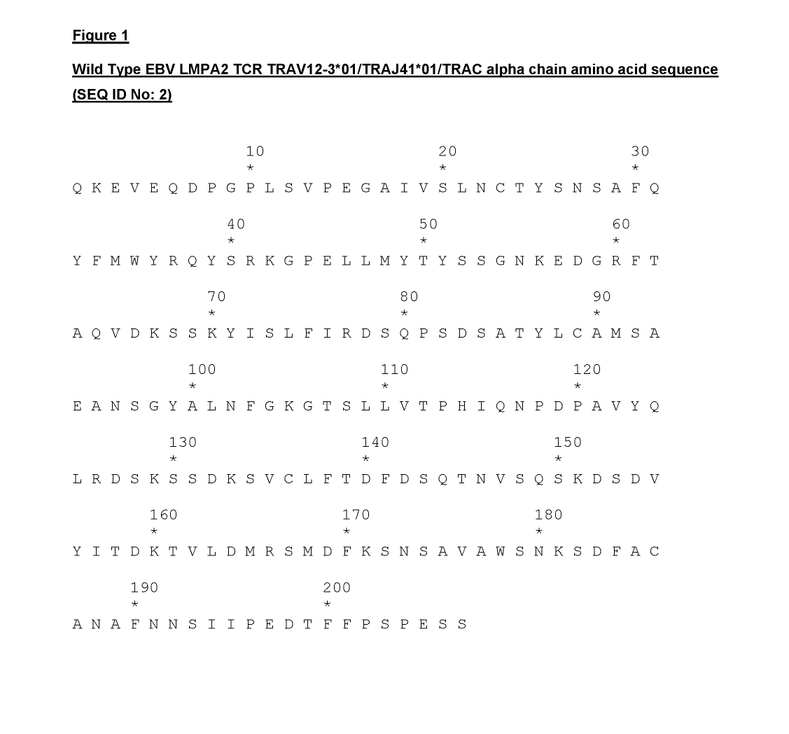

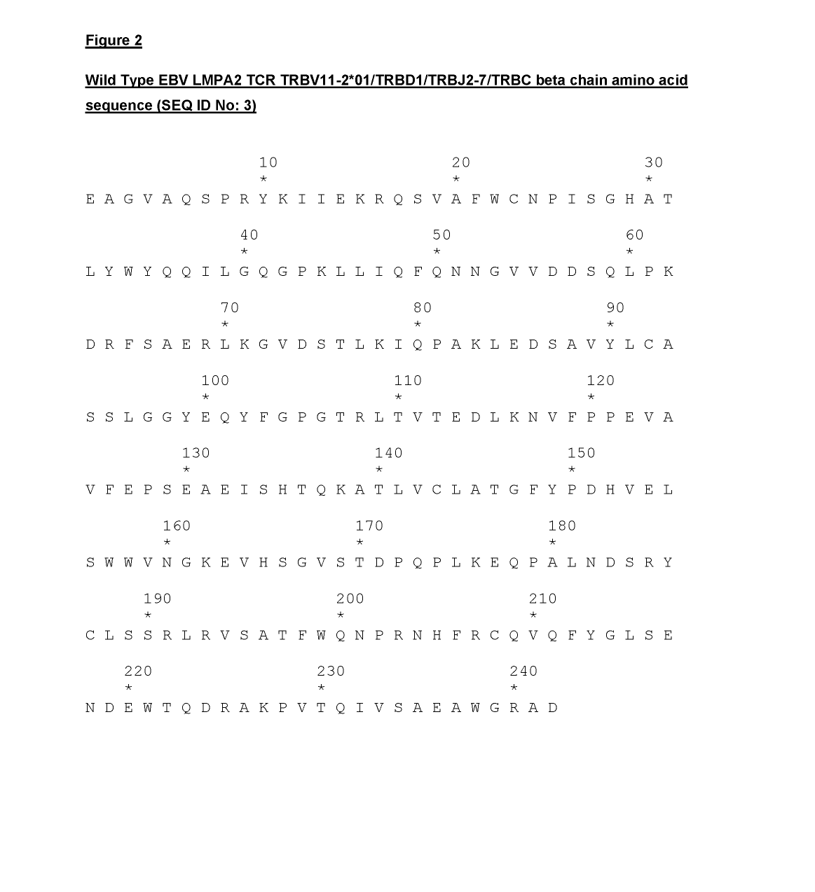

### DETAILED DESCRIPTION OF THE INVENTION

According to a first aspect of the invention, there is provided a T cell receptor (TCR) having the property of binding to CLGGLLTMV (SEQ ID No: 1) HLA-A2 complex and comprising a TCR alpha chain variable domain and/or a TCR beta chain variable domain,
the alpha chain variable domain comprising an amino acid sequence that has at least 80% identity to the sequence of amino acid residues 1-113 of SEQ ID No: 2, and/or
the beta chain variable domain comprising an amino acid sequence that has at least 80% identity to the sequence of amino acid residues 1-112 of SEQ ID No: 3,
wherein the alpha chain variable domain has at least one of the following mutations:

| Residue no. | | |
|---|---|---|
| 28 | P | |
| 29 | H | |
| 30 | M | |
| 31 | A | |
| 50 | L | Q |
| 51 | P | F |
| 52 | G | Q |
| 53 | G | D |
| 94 | D | |
| 96 | Y | H |
| 97 | G | Q |
| 98 | H | P |
| 100 | R | |

and/or the beta chain variable domain has at least one of the following mutations:

| Residue no. | |
|---|---|
| 50 | V |
| 51 | V |
| 52 | A |
| 53 | A |
| 54 | S |

TCRs of the invention may be non-naturally occurring and/or purified and/or engineered. TCRs of the invention may have more than one mutation present in the alpha chain variable domain and/or the beta chain variable domain. In certain embodiments, there are 2-20, 3-15, 4-12 or 4-10 mutations in one or both variable domains. There may be 2, 3, 4, 5, 6, 7, 8, 9, 10, 11, 12, 13, 14, 15, 16, 17, 18, 19, 20, 21 or 22 mutations in one or both variable domains. In some embodiments, the α chain variable domain of the TCR of the invention may comprise an amino acid sequence that has at least 90%, at least 91%, at least 92%, at least 93%, at least 94%, at least 95%, at least 96%, at least 97%, at least 98% or at least 99% identity to the sequence of amino acid residues 1-113 of SEQ ID No: 2, provided that the α chain variable domain has at least one of the mutations outlined above. In some embodiments, the β chain variable domain of the TCR of the invention may comprise an amino acid sequence that has at least 90%, at least 91%, at least 92%, at least 93%, at least 94%, at least 95%, at least 96%, at least 97%, at least 98% or at least 99% identity to the sequence of amino acid residues 1-112 of SEQ ID No: 3, provided that the β chain variable domain has at least one of the mutations outlined above.

Also within the scope of the invention are phenotypically silent variants of any TCR disclosed herein. As used herein the term “phenotypically silent variants” is understood to refer to those TCRs which have a K_{D} and/or binding half-life for the CLGGLLTMV (SEQ ID No: 1) HLA-A2 complex within the ranges of K_{D}s and binding half-lives described below. For example, as is known to those skilled in the art, it may be possible to produce TCRs that incorporate changes in the constant and/or variable domains thereof compared to those detailed above without altering the affinity for the interaction with the CLGGLLTMV (SEQ ID No: 1) HLA-A2 complex. Such trivial variants are included in the scope of this invention. Those TCRs in which one or more conservative substitutions have been made also form part of this invention.

Mutations can be carried out using any appropriate method including, but not limited to, those based on polymerase chain reaction (PCR), restriction enzyme-based cloning, or ligation independent cloning (LIC) procedures. These methods are detailed in many of the of the standard molecular biology texts. For further details regarding polymerase chain reaction (PCR) and restriction enzyme-based cloning, see Sambrook & Russell, (2001) Molecular Cloning—A Laboratory Manual (3^{rd} Ed.) CSHL Press. Further information on ligation independent cloning (LIC) procedures can be found in Rashtchian, (1995) Curr Opin Biotechnol 6(1): 30-6.

The TCRs of the invention have the property of binding the CLGGLLTMV (SEQ ID No: 1) HLA-A2 complex, or natural variants SLGGLLTMV (SEQ ID No: 17) and CLGGLITMV (SEQ ID No: 18). Certain TCRs of the invention have been found to be highly specific for these epitopes relative to other, irrelevant epitopes, and are thus particularly suitable as targeting vectors for delivery of therapeutic agents or detectable labels to cells and tissues displaying those epitopes. Specificity in the context of TCRs of the invention relates to their ability to recognise EBV LMP2A antigen positive HLA-A2 positive target cells whilst having minimal ability to recognise EBV LMP2A negative targets cells, particularly non-cancerous human cells. Specificity can be measured, for example, in cellular assays such as those described in Example 6. Certain TCRs of the invention have been found to be highly suitable for use in adoptive therapy. Such TCRs may have a K_{D} for the complex of less than 23 μM, for example from about 0.1 μM to about 22 μM and/or have a binding half-life (T½) for the complex in the range of from about 3 seconds to about 12 minutes. In some embodiments, TCRs of the invention may have a K_{D} for the complex of from about 0.5 μM to about 15 μM, about 1 μM to about 10 μM or about 2 μM to about 5 The TCR of the invention may have a K_{D} for the complex of about 3 Certain TCRs of the invention have been found to be highly suitable for use as therapeutics and/or diagnostics when coupled to a detectable label or therapeutic agent. Such TCRs may have a K_{D} for the complex in the range of from about 10 pM to about 200 nM and a T½ of about 10 minutes to about 60 hours. In some embodiments, TCRs of the invention may have a K_{D} for the complex of from about 12 pM to about 100 nM, from about 15 pM to about 1 nM, from about 20 pM to about 0.5 nM, from about 30 pM, to about 0.1 nM or from about 40 pM to about 50 pM.

For use as a targeting agent for delivering therapeutic agents to the antigen presenting cell the TCR may be in soluble form (i.e. having no transmembrane or cytoplasmic domains). For stability, TCRs of the invention, and preferably soluble αβ heterodimeric TCRs, may have an introduced disulfide bond between residues of the respective constant domains, as described, for example, in WO 03/020763. One or both of the constant domains present in an αβ heterodimer of the invention may be truncated at the C terminus or C termini, for example by up to 15, or up to 10 or up to 8 or fewer amino acids. For use in adoptive therapy, an αβ heterodimeric TCR may, for example, be transfected as full length chains having both cytoplasmic and transmembrane domains.

The TCRs of the invention may be αβ heterodimers or may be in single chain format. Single chain formats include αβ TCR polypeptides of the Vα-L-Vβ, Vβ-L-Vα, Vα-Cα-L-Vβ, or Vα-L-Vβ-Cβ types, wherein Vα and Vβ are TCR α and β variable regions respectively, Cα and Cβ are TCR α and β constant regions respectively, and L is a linker sequence. In certain embodiments single chain TCRs of the invention may have an introduced disulfide bond between residues of the respective constant domains, as described in WO 2004/033685.

In some embodiments, the alpha chain variable domain may have at least 96, 97, 98 or 99% sequence identity, or 100% sequence identity, to the amino acid sequence from Q1 to P113 of SEQ ID No: 6 or of SEQ ID No: 7 or of SEQ ID No: 8 or of SEQ ID No: 9 or of SEQ ID No: 10 or of SEQ ID No: 11 or of SEQ ID No: 12. The amino acids underlined in FIG. 5 may be invariant.

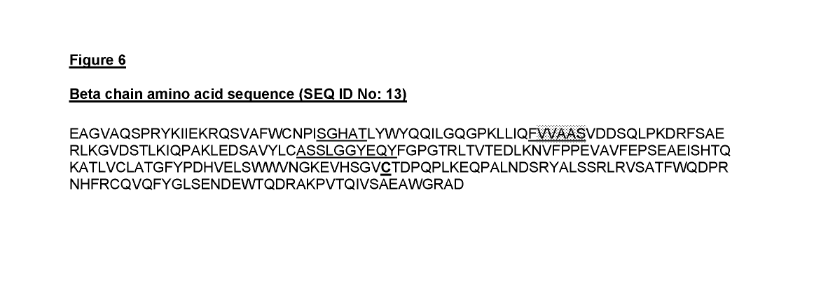

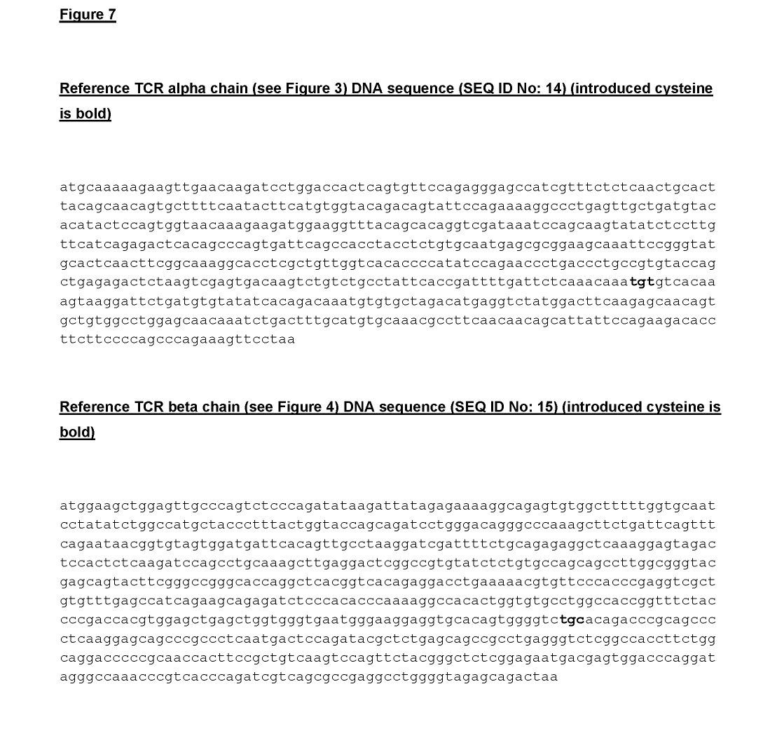

In some embodiments, the beta chain variable domain may have at least 96, 97, 98 or 99% sequence identity, or 100% sequence identity, to the amino acid sequence from E1 to T112 of SEQ ID No: 13. The amino acids underlined in FIG. 6 may be invariant.

In one subclass of TCRs of the invention, the alpha chain variable domain may comprise Q1 to P113 of SEQ ID No: 6 or of SEQ ID No: 7 or of SEQ ID No: 8 or of SEQ ID No: 9 or of SEQ ID No: 10 or of SEQ ID No: 11 or of SEQ ID No: 12; and/or the beta chain may comprise E1 to T112 of SEQ ID No: 13. A TCR of the invention may comprise an alpha chain variable domain comprising Q1 to P113 of SEQ ID No: 9 and a beta chain comprising E1 to T112 of SEQ ID No: 13. A TCR of the invention may comprise an alpha chain variable domain comprising Q1 to P113 of SEQ ID No: 7 and a beta chain comprising E1 to T112 of SEQ ID No: 13.

As will be obvious to those skilled in the art, it may be possible to truncate the sequences provided at the C-terminus and/or N-terminus thereof, by 1, 2, 3, 4, 5 or more residues, without substantially affecting the binding characteristics of the TCR. All such trivial variants are encompassed by the present invention.

Alpha-beta heterodimeric TCRs of the invention usually comprise an alpha chain TRAC constant domain sequence and/or a beta chain TRBC1 or TRBC2 constant domain sequence. The alpha and beta chain constant domain sequences may be modified by truncation or substitution to delete the native disulfide bond between Cys4 of exon 2 of TRAC and Cys2 of exon 2 of TRBC1 or TRBC2. The alpha and/or beta chain constant domain sequence(s) may also be modified by substitution of cysteine residues for Thr 48 of TRAC and Ser 57 of TRBC1 or TRBC2, the said cysteines forming a disulfide bond between the alpha and beta constant domains of the TCR.

Certain TCRs of the invention have a binding affinity for, and/or a binding half-life for, the CLGGLLTMV-HLA-A2 complex (“CLGGLLTMV” disclosed as SEQ ID NO: 1) complex substantially higher than that of the reference EBV LMP2A TCR. Increasing the binding affinity of a native TCR often reduces the selectivity of the TCR for its peptide-MHC ligand, and this is demonstrated in Zhao Yangbing et al., The Journal of Immunology, The American Association of Immunologists, US, vol. 179, No. 9, 1 Nov. 2007, 5845-5854. However, the TCRs of the invention remain selective for the CLGGLLTMV-HLA-A2 complex (“CLGGLLTMV” disclosed as SEQ ID NO: 1), despite, in some embodiments, having substantially higher binding affinity than the parent native TCR.

Binding affinity (inversely proportional to the equilibrium constant K_{D}) and binding half-life (expressed as T½) can be determined by any appropriate method. It will be appreciated that doubling the affinity of a TCR results in halving the K_{D}. T½ is calculated as ln 2 divided by the off-rate (k_{off}). Therefore, doubling of T½ results in a halving in k_{off}. K_{D} and k_{off} values for TCRs are usually measured for soluble forms of the TCR, i.e. those forms which are truncated to remove cytoplasmic and transmembrane domain residues. Therefore it is to be understood that a given TCR meets the requirement that it has a binding affinity for, and/or a binding half-life for, the CLGGLLTMV-HLA-A2 complex (“CLGGLLTMV” disclosed as SEQ ID NO: 1) if a soluble form of that TCR meets that requirement. Preferably the binding affinity or binding half-life of a given TCR is measured several times, for example 3 or more times, using the same assay protocol and an average of the results is taken. In a preferred embodiment these measurements are made using the Surface Plasmon Resonance (BIAcore) method of Example 3 herein. The reference CLGGLLTMV-HLA-A2 TCR (“CLGGLLTMV” disclosed as SEQ ID NO: 1) has a K_{D} of approximately 23 μM as measured by that method, and its T½ is approximately 3 s.

In a further aspect, the present invention provides nucleic acid encoding a TCR of the invention. In some embodiments, the nucleic acid is cDNA. In some embodiments, the invention provides nucleic acid comprising a sequence encoding an α chain variable domain of a TCR of the invention. In some embodiments, the invention provides nucleic acid comprising a sequence encoding a β chain variable domain of a TCR of the invention. The nucleic acid may be non-naturally occurring and/or purified and/or engineered.

In another aspect, the invention provides a vector which comprises nucleic acid of the invention. Preferably the vector is a TCR expression vector.

The invention also provides a cell harbouring a vector of the invention, preferably a TCR expression vector. The vector may comprise nucleic acid of the invention encoding in a single open reading frame, or two distinct open reading frames, the alpha chain and the beta chain respectively. Another aspect provides a cell harbouring a first expression vector which comprises nucleic acid encoding the alpha chain of a TCR of the invention, and a second expression vector which comprises nucleic acid encoding the beta chain of a TCR of the invention. Such cells are particularly useful in adoptive therapy. The cells of the invention may be isolated and/or recombinant and/or non-naturally occurring and/or engineered.

Since the TCRs of the invention have utility in adoptive therapy, the invention includes a non-naturally occurring and/or purified and/or or engineered cell, especially a T-cell, presenting a TCR of the invention. There are a number of methods suitable for the transfection of T cells with nucleic acid (such as DNA, cDNA or RNA) encoding the TCRs of the invention (see for example Robbins et al., (2008) J Immunol. 180: 6116-6131). T cells expressing the TCRs of the invention will be suitable for use in adoptive therapy-based treatment of EBV infection. As will be known to those skilled in the art, there are a number of suitable methods by which adoptive therapy can be carried out (see for example Rosenberg et al., (2008) Nat Rev Cancer 8(4): 299-308).

Some soluble TCRs of the invention are useful for delivering detectable labels or therapeutic agents to antigen presenting cells and tissues containing antigen presenting cells. They may therefore be associated (covalently or otherwise) with a detectable label (for diagnostic purposes wherein the TCR is used to detect the presence of cells presenting the CLGGLLTMV-HLA-A2 complex (“CLGGLLTMV” disclosed as SEQ ID NO: 1)); a therapeutic agent; or a PK modifying moiety (for example by PEGylation).

Detectable labels for diagnostic purposes include for instance, fluorescent labels, radiolabels, enzymes, nucleic acid probes and contrast reagents.

Therapeutic agents which may be associated with the TCRs of the invention include immunomodulators, radioactive compounds, enzymes (perforin for example) or chemotherapeutic agents (cis-platin for example). To ensure that toxic effects are exercised in the desired location the toxin could be inside a liposome linked to TCR so that the compound is released slowly. This will prevent damaging effects during the transport in the body and ensure that the toxin has maximum effect after binding of the TCR to the relevant antigen presenting cells.

Other suitable therapeutic agents include:
small molecule cytotoxic agents, i.e. compounds with the ability to kill mammalian cells having a molecular weight of less than 700 Daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate glucuronate, auristatin E vincristine and doxorubicin;
peptide cytotoxins, i.e. proteins or fragments thereof with the ability to kill mammalian cells. For example, ricin, diphtheria toxin, pseudomonas bacterial exotoxin A, DNase and RNase;
radio-nuclides, i.e. unstable isotopes of elements which decay with the concurrent emission of one or more of α or β particles, or γ rays. For example, iodine 131, rhenium 186, indium 111, yttrium 90, bismuth 210 and 213, actinium 225 and astatine 213; chelating agents may be used to facilitate the association of these radio-nuclides to the high affinity TCRs, or multimers thereof;
immuno-stimulants, i.e. immune effector molecules which stimulate immune response. For example, cytokines such as IL-2 and IFN-γ,
Superantigens and mutants thereof;
TCR-HLA fusions;
chemokines such as IL-8, platelet factor 4, melanoma growth stimulatory protein, etc;
antibodies or fragments thereof, including anti-T cell or NK cell determinant antibodies (e.g. anti-CD3, anti-CD28 or anti-CD16);
alternative protein scaffolds with antibody like binding characteristics
complement activators;
xenogeneic protein domains, allogeneic protein domains, viral/bacterial protein domains, viral/bacterial peptides.

One preferred embodiment is provided by a TCR of the invention associated (usually by fusion to an N- or C-terminus of the alpha or beta chain) with an anti-CD3 antibody, or a functional fragment or variant of said anti-CD3 antibody. As used herein, the term “antibody” encompasses such fragments and variants. Examples of anti-CD3 antibodies include but are not limited to OKT3, UCHT-1, BMA-031 and 12F6. Antibody fragments and variants/analogues which are suitable for use in the compositions and methods described herein include minibodies, Fab fragments, F(ab′)₂ fragments, dsFv and scFv fragments, Nanobodies™ (these constructs, marketed by Ablynx (Belgium), comprise synthetic single immunoglobulin variable heavy domain derived from a camelid (e.g. camel or llama) antibody) and Domain Antibodies (Domantis (Belgium), comprising an affinity matured single immunoglobulin variable heavy domain or immunoglobulin variable light domain) or alternative protein scaffolds that exhibit antibody like binding characteristics such Affibodies (Affibody (Sweden), comprising engineered protein A scaffold) or Anticalins (Pieris (Germany)), comprising engineered anticalins) to name but a few.

Linkage of the TCR and the anti-CD3 antibody may be direct, or indirect via a linker sequence. Linker sequences are usually flexible, in that they are made up primarily of amino acids such as glycine, alanine and serine which do not have bulky side chains likely to restrict flexibility. Usable or optimum lengths of linker sequences are easily determined. Often the linker sequence will be less than about 12, such as less than 10, or from 5-10 amino acids in length. Suitable linkers that may be used in TCRs of the invention include, but are not limited to: GGGGS (SEQ ID No: 23), GGGSG (SEQ ID No: 24), GGSGG (SEQ ID No: 25), GSGGG (SEQ ID No: 26), GSGGGP (SEQ ID No: 27), GGEPS (SEQ ID No: 28), GGEGGGP (SEQ ID No: 29), and GGEGGGSEGGGS (SEQ ID No: 30) (as described in WO2010/133828).

Specific embodiments of anti-CD3-TCR fusion constructs of the invention include those which have an alpha chain variable domain selected from SEQ ID No: 6, or SEQ ID No: 7, or SEQ ID No: 8, or SEQ ID No: 9, or SEQ ID No: 10, or SEQ ID No: 11 or SEQ ID No: 12, and the TCR beta chain SEQ ID No: 13 fused to an amino acid sequence corresponding to anti-CD3. The amino acid at position 1 of the alpha chain sequences may be replaced with an alternative amino acid selected from A and G.

More particularly, TCR-anti CD3 fusions of the invention may include a TCR alpha chain amino acid sequence selected from the group consisting of:
(i) the TCR alpha chain sequence SEQ ID No: 2 or SEQ ID No: 4, wherein amino acids 1 to 113 are replaced by the amino acids 1-113 of sequence SEQ ID No: 9;
(ii) the TCR alpha chain sequence SEQ ID No: 2 or SEQ ID No: 4, wherein amino acids 1 to 113 are replaced by the amino acids 1-113 of sequence SEQ ID No: 9, wherein the amino acid at position 1 is replaced by A;
(iii) the TCR alpha chain sequence SEQ ID No: 2 or SEQ ID No: 4, wherein amino acids 1 to 113 are replaced by the amino acids 1-113 of sequence SEQ ID No: 9, wherein the amino acid at position 1 is replaced by G;
(iv) the TCR alpha chain sequence SEQ ID No: 2 or SEQ ID No: 4, wherein amino acids 1 to 113 are replaced by the amino acids 1-113 of sequence SEQ ID No: 9, and the C-terminus of the alpha chain is truncated by 8 amino acids from F200 to S207 inclusive, based on the numbering of SEQ ID No: 4;
(v) the TCR alpha chain sequence SEQ ID No: 2 or SEQ ID No: 4, wherein amino acids 1 to 113 are replaced by the amino acids 1-113 of sequence SEQ ID No: 9, wherein the amino acid at position 1 is replaced by A, and the C-terminus of the alpha chain is truncated by 8 amino acids from F200 to S207 inclusive, based on the numbering of SEQ ID No: 4;
(vi) the TCR alpha chain sequence SEQ ID No: 2 or SEQ ID No: 4, wherein amino acids 1 to 113 are replaced by the amino acids 1-113 of sequence SEQ ID No: 9, wherein the amino acid at position 1 is replaced by G, and the C-terminus of the alpha chain is truncated by 8 amino acids from F200 to S207 inclusive, based on the numbering of SEQ ID No: 4.
and/or a TCR beta chain-anti-CD3 amino acid sequence selected from the group consisting of:(vii)
the TCR beta chain-anti-CD3 sequence SEQ ID No: 16, wherein amino acids at positions 1 and 2 are D and I respectively;
(viii) the TCR beta chain-anti-CD3 sequence SEQ ID No: 16, wherein amino acids at positions 1 and 2 are A and I respectively;
(ix) the TCR beta chain-anti-CD3 sequence SEQ ID No: 16, wherein amino acids at positions 1 and 2 are A and Q respectively;
(x) the TCR beta chain-anti-CD3 sequence SEQ ID No: 16, wherein amino acids at positions 1 and 2 are D and I respectively, amino acids at positions 108-131 are replaced by RTSGPGDGGKGGPGKGPGGEGTKGTGPGG (SEQ ID No: 31), and amino acids at positions 254-258 are replaced by GGEGGGSEGGGS (SEQ ID No: 30);
(xi) the TCR beta chain-anti-CD3 sequence SEQ ID No: 16, wherein amino acids at positions 1 and 2 are D and I respectively, the amino acid at position 257 is S and the amino acid at position 258 is G;
(xii) the TCR beta chain-anti-CD3 sequence SEQ ID No: 16, wherein amino acids at positions 1 and 2 are D and I respectively, the amino acid at position 256 is S and the amino acid at position 258 is G;
(xiii) the TCR beta chain-anti-CD3 sequence SEQ ID No: 16, wherein amino acids at positions 1 and 2 are D and I respectively, the amino acid at position 255 is S and the amino acid at position 258 is G;
(xiv) a TCR beta chain-anti-CD3 having the sequence SEQ ID No: 16, wherein amino acids at positions 1 and 2 are A and Q, the amino acid at position 257 is S and the amino acid at position 258 is G.
(xv) a TCR beta chain-anti-CD3 having the sequence SEQ ID No: 16, wherein amino acids at positions 1 and 2 are A and Q, the amino acid at position 256 is S and the amino acid at position 258 is G;
(xvi) a TCR beta chain-anti-CD3 having the sequence SEQ ID No: 16, wherein amino acids at positions 1 and 2 are A and Q, the amino acid at position 255 is S and the amino acid at position 258 is G;
(xvii) and a TCR beta chain-anti-CD3 having the sequence SEQ ID No: 16, wherein amino acid at positions 1 and 2 are A and I respectively, the amino acid at position 257 is S and the amino acid at position 258 is G;
(xviii) a TCR beta chain-anti-CD3 having the sequence SEQ ID No: 16, wherein amino acid at positions 1 and 2 are A and I respectively, the amino acid at position 256 is S and the amino acid at position 258 is G;
(xix) a TCR beta chain-anti-CD3 having the sequence SEQ ID No: 16, wherein amino acid at positions 1 and 2 are A and I respectively, the amino acid at position 255 is S and the amino acid at position 258 is G.

With reference to the above, examples of such TCR-anti CD3 fusions are:
a TCR-anti CD3 fusion in which the alpha chain amino acid sequence is (i) and the beta chain-anti-CD3 amino acid sequence is (vii);
a TCR-anti CD3 fusion in which the alpha chain amino acid sequence is (i) and the beta chain-anti-CD3 amino acid sequence is (x);
a TCR-anti CD3 fusion in which the alpha chain amino acid sequence is (vi) and the beta chain-anti-CD3 amino acid sequence is (ix);
a TCR-anti CD3 fusion in which the alpha chain amino acid sequence is (v) and the beta chain-anti-CD3 amino acid sequence is (viii);
a TCR-anti CD3 fusion in which the alpha chain amino acid sequence is (vi) and the beta chain-anti-CD3 amino acid sequence is (vii);
a TCR-anti CD3 fusion in which the alpha chain amino acid sequence is (i) and the beta chain-anti-CD3 amino acid sequence is (xi);
a TCR-anti CD3 fusion in which the alpha chain amino acid sequence is (i) and the beta chain-anti-CD3 amino acid sequence is (xii);
a TCR-anti CD3 fusion in which the alpha chain amino acid sequence is (i) and the beta chain-anti-CD3 amino acid sequence is (xiii);
a TCR-anti CD3 fusion in which the alpha chain amino acid sequence is (vi) and the beta chain-anti-CD3 amino acid sequence is (xiv);
a TCR-anti CD3 fusion in which the alpha chain amino acid sequence is (vi) and the beta chain-anti-CD3 amino acid sequence is (xv);
a TCR-anti CD3 fusion in which the alpha chain amino acid sequence is (vi) and the beta chain-anti-CD3 amino acid sequence is (xvi);
a TCR-anti CD3 fusion in which the alpha chain amino acid sequence is (v) and the beta chain-anti-CD3 amino acid sequence is (xvii);
a TCR-anti CD3 fusion in which the alpha chain amino acid sequence is (v) and the beta chain-anti-CD3 amino acid sequence is (xviii);
a TCR-anti CD3 fusion in which the alpha chain amino acid sequence is (v) and the beta chain-anti-CD3 amino acid sequence is (xix);
a TCR-anti CD3 fusion in which the alpha chain amino acid sequence is (vi) and the beta chain-anti-CD3 amino acid sequence is (xi);
a TCR-anti CD3 fusion in which the alpha chain amino acid sequence is (vi) and the beta chain-anti-CD3 amino acid sequence is (xii); and a TCR-anti CD3 fusion in which the alpha chain amino acid sequence is (vi) and the beta chain-anti-CD3 amino acid sequence is (xiii).

For some purposes, the TCRs of the invention may be aggregated into a complex comprising several TCRs to form a multivalent TCR complex. There are a number of human proteins that contain a multimerisation domain that may be used in the production of multivalent TCR complexes. For example the tetramerisation domain of p53 which has been utilised to produce tetramers of scFv antibody fragments which exhibited increased serum persistence and significantly reduced off-rate compared to the monomeric scFv fragment (Willuda et al. (2001) J. Biol. Chem. 276 (17) 14385-14392). Haemoglobin also has a tetramerisation domain that could be used for this kind of application. A multivalent TCR complex of the invention may have enhanced binding capability for the CLGGLLTMV-HLA-A2 complex (“CLGGLLTMV” disclosed as SEQ ID NO: 1) compared to a non-multimeric wild-type or T cell receptor heterodimer of the invention. Thus, multivalent complexes of TCRs of the invention are also included within the invention. Such multivalent TCR complexes according to the invention are particularly useful for tracking or targeting cells presenting particular antigens in vitro or in vivo, and are also useful as intermediates for the production of further multivalent TCR complexes having such uses.

As is well-known in the art, TCRs may be subject to post translational modifications. Glycosylation is one such modification, which comprises the covalent attachment of oligosaccharide moieties to defined amino acids in the TCR chain. For example, asparagine residues, or serine/threonine residues are well-known locations for oligosaccharide attachment. The glycosylation status of a particular protein depends on a number of factors, including protein sequence, protein conformation and the availability of certain enzymes. Furthermore, glycosylation status (i.e oligosaccharide type, covalent linkage and total number of attachments) can influence protein function. Therefore, when producing recombinant proteins, controlling glycosylation is often desirable. Controlled glycosylation has been used to improve antibody based therapeutics. (Jefferis R., Nat Rev Drug Discov. 2009 March; 8(3):226-34.).

For soluble TCRs of the invention glycosylation may be controlled in vivo, by using particular cell lines for example, or in vitro, by chemical modification. Such modifications are desirable, since glycosylation can improve phamacokinetics, reduce immunogenicity and more closely mimic a native human protein (Sinclair A M and Elliott S., Pharm Sci. 2005 August; 94(8): 1626-35).

For administration to patients, the TCRs of the invention (preferably associated with a detectable label or therapeutic agent or expressed on a transfected T cell) or cells of the invention may be provided in a pharmaceutical composition together with one or more pharmaceutically acceptable carriers or excipients. Therapeutic or imaging TCRs, or cells, in accordance with the invention will usually be supplied as part of a sterile, pharmaceutical composition which will normally include a pharmaceutically acceptable carrier. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, preferably a parenteral (including subcutaneous, intramuscular, or preferably intravenous) route. Such compositions may be prepared by any method known in the art of pharmacy, for example by mixing the active ingredient with the carrier(s) or excipient(s) under sterile conditions.

Dosages of the substances of the present invention can vary between wide limits, depending upon the disease or disorder to be treated, the age and condition of the individual to be treated, etc. a suitable dose range for an soluble TCR of the invention associated with an anti-CD3 antibody may be between 25 ng/kg and 50 μg/kg. A physician will ultimately determine appropriate dosages to be used.

TCRs, pharmaceutical compositions, vectors, nucleic acids and cells of the invention may be provided in substantially pure form, for example at least 80%, at least 85%, at least 90%, at least 91%, at least 92%, at least 93%, at least 94%, at least 95%, at least 96%, at least 97%, at least 98%, at least 99% or 100% pure.

Also provided by the invention are:
a TCR which binds the CLGGLLTMV peptide (SEQ ID NO: 1) (derived from the EBV LMP2A protein) presented as a peptide-HLA-A2 complex, or a cell expressing and/or presenting such a TCR, for use in medicine, preferably in a method of treating EBV infection. The TCR may be non-naturally occurring and/or purified and/or engineered;
the use of a TCR which binds the CLGGLLTMV peptide (SEQ ID NO: 1) (derived from the EBV LMP2A protein) presented as a peptide-HLA-A1 complex, or a cell expressing and/or presenting such a TCR, in the manufacture of a medicament for treating EBV infection;
a method of treating EBV infection in a patient, comprising administering to the patient a TCR which binds the CLGGLLTMV peptide (SEQ ID NO: 1) (derived from the EBV LMP2A protein) presented as a peptide-HLA-A2 complex, or a cell expressing and/or presenting such a TCR.

It is preferred that the TCR which binds the CLGGLLTMV peptide (SEQ ID NO: 1) (derived from the EBV LMP2A protein) presented as a peptide-HLA-A2 complex is a TCR of the invention.

Preferred features of each aspect of the invention are as for each of the other aspects mutatis mutandis. The prior art documents mentioned herein are incorporated to the fullest extent permitted by law.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### Cloning of the Reference EBV LMP2A TCR Alpha and Beta Chain Variable Region Sequences into pGMT7-Based Expression Plasmids

The reference EBV LMP2A TCR variable alpha and TCR variable beta domains were PCR amplified from total cDNA isolated from an EBV LMP2A T cell clone (Clone SB34 from John Miles and Scott Burrows, Queensland Institute of Medical Research, Brisbane, Australia). In the case of the alpha chain, an alpha chain variable region sequence specific oligonucleotide A1 gaattccatatgcaaaaagaagttgaacaagatcctggaccactc (SEQ ID No: 19) which encodes the restriction site NdeI and an alpha chain constant region sequence specific oligonucleotide A2 ttgtcagtcgacttagagtctctcagctggtacacg (SEQ ID No:20) which encodes the restriction site SalI are used to amplify the alpha chain variable domain. In the case of the beta chain, a beta chain variable region sequence specific oligonucleotide B1 gaattccatatggaagctggagttgctcaatctcccagatataag (SEQ ID No:21) which encodes the restriction site NdeI and a beta chain constant region sequence specific oligonucleotide B2 tagaaaccggtggccaggcacaccagtgtggc (SEQ ID No:22) which encodes the restriction site Age1 are used to amplify the beta chain variable domain.

The alpha and beta variable domains were cloned into pGMT7-based expression plasmids containing either Cα or Cβ by standard methods described in (Molecular Cloning a Laboratory Manual Third edition by Sambrook and Russell). Plasmids were sequenced using an Applied Biosystems 3730×1 DNA Analyzer.

The DNA sequences encoding the TCR alpha chain cut with NdeI and SalI were ligated into pGMT7+Cα vector, which was cut with NdeI and XhoI. The DNA sequences encoding the TCR beta chain cut with NdeI and AgeI was ligated into separate pGMT7+Cβ vector, which was also cut with NdeI and AgeI.

### Ligation

Ligated plasmids were transformed into competent E. coli strain XL1-blue cells and plated out on LB/agar plates containing 100 μm/ml ampicillin. Following incubation overnight at 37° C., single colonies are picked and grown in 10 ml LB containing 100 μg/ml ampicillin overnight at 37° C. with shaking. Cloned plasmids were purified using a Miniprep kit (Qiagen) and the plasmids were sequenced using an Applied Biosystems 3730×1 DNA Analyzer.

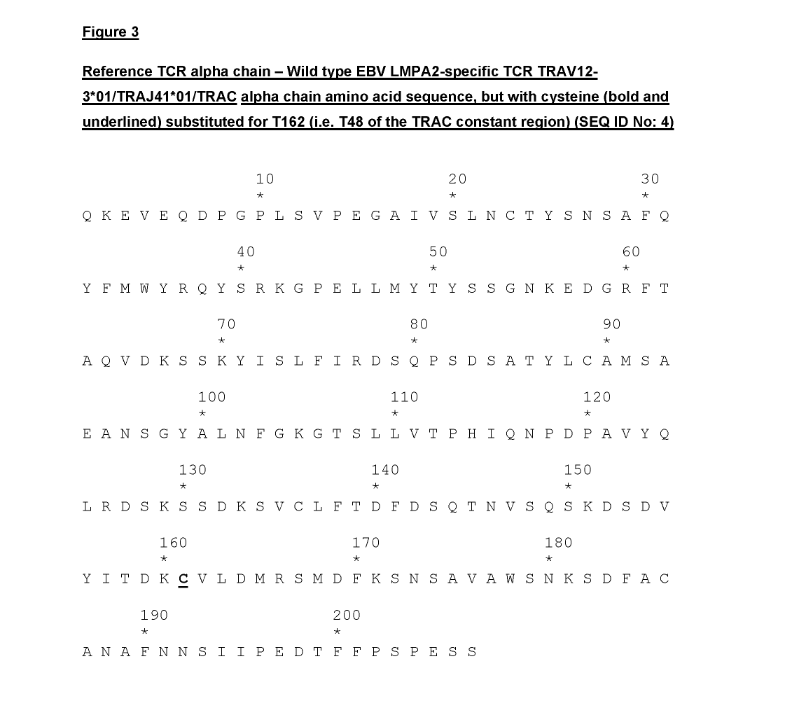

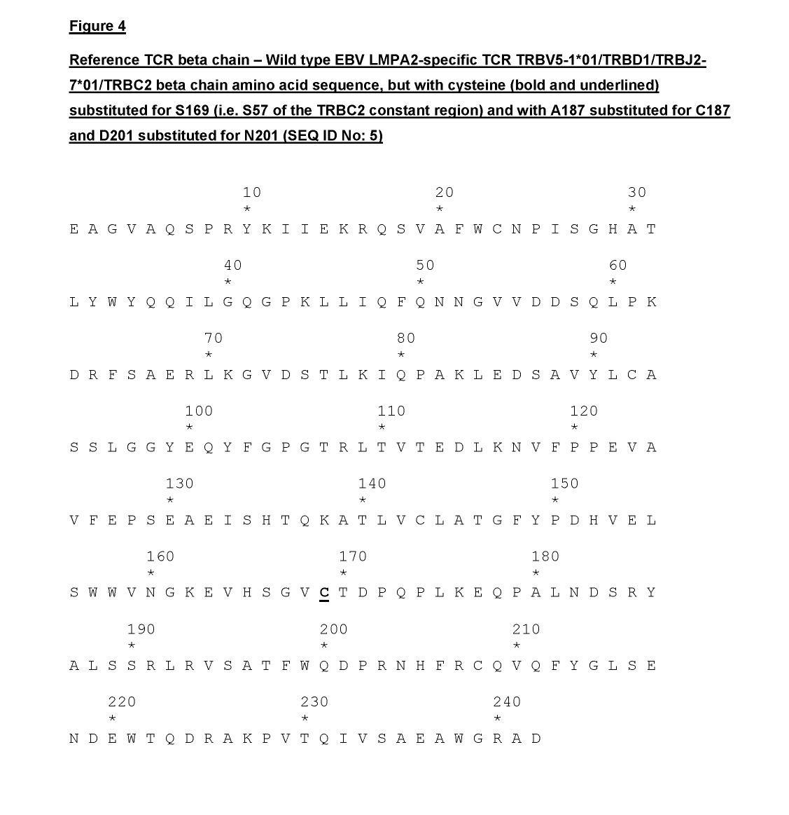

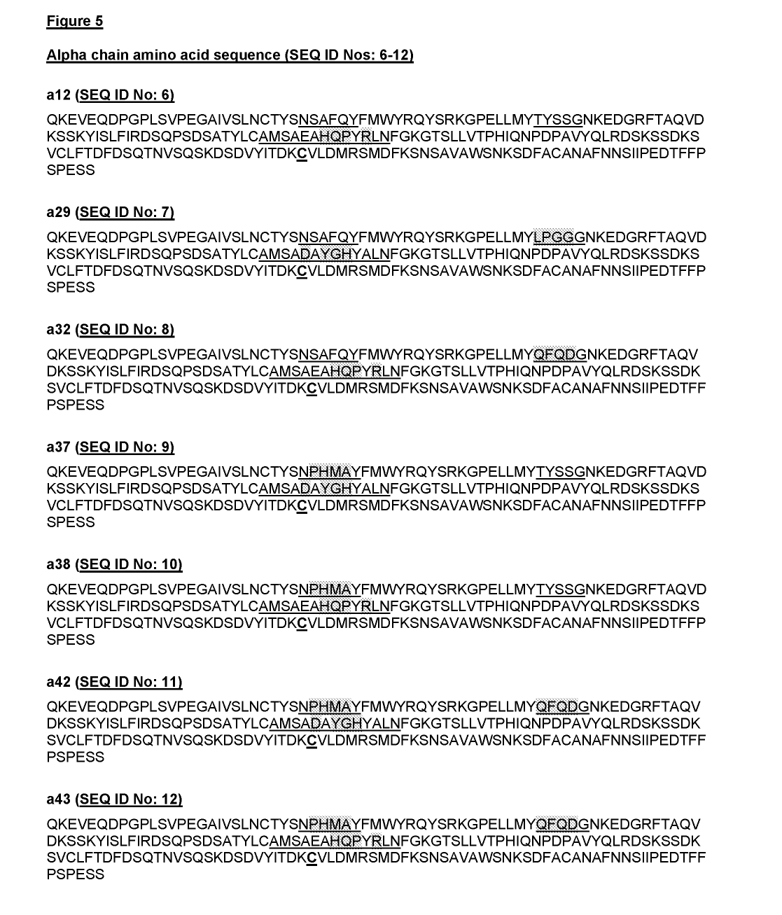

FIGS. 3 and 4 show respectively the reference EBV LMP2A TCR α and β chain extracellular amino acid sequences (SEQ ID Nos: 4 and 5 respectively) produced from the DNA sequences of FIG. 7 (SEQ ID No: 14) (SEQ ID No: 15) respectively. Note that, relative to the native TCR, cysteines were substituted in the constant regions of the alpha and beta chains to provide an artificial inter-chain disulphide bond on refolding to form the heterodimeric TCR. The introduced cysteines are shown in bold and underlined. The restriction enzyme recognition sequences in the DNA sequences of FIG. 7 are underlined.

### Example 2

### Expression, Refolding and Purification of Soluble Reference EBV LMP2A TCR

The expression plasmids containing the TCR α-chain and β-chain respectively, as prepared in Example 1, were transformed separately into E. coli strain BL21pLysS, and single ampicillin-resistant colonies were grown at 37° C. in TYP (ampicillin 100 μg/ml) medium to OD₆₀₀ of ˜0.6-0.8 before inducing protein expression with 0.5 mM IPTG. Cells were harvested three hours post-induction by centrifugation for 30 minutes at 4000 rpm in a Beckman J-6B. Cell pellets were lysed with 25 ml Bug Buster (NovaGen) in the presence of MgCl₂ and DNaseI. Inclusion body pellets were recovered by centrifugation for 30 minutes at 13000 rpm in a Beckman J2-21 centrifuge. Three detergent washes were then carried out to remove cell debris and membrane components. Each time the inclusion body pellet was homogenised in a Triton buffer (50 mM Tris-HCl pH 8.0, 0.5% Triton-X100, 200 mM NaCl, 10 mM NaEDTA) before being pelleted by centrifugation for 15 minutes at 13000 rpm in a Beckman J2-21. Detergent and salt was then removed by a similar wash in the following buffer: 50 mM Tris-HCl pH 8.0, 1 mM NaEDTA. Finally, the inclusion bodies were divided into 30 mg aliquots and frozen at −70° C. Inclusion body protein yield was quantified by solubilising with 6 M guanidine-HCl and an OD measurement was taken on a Hitachi U-2001 Spectrophotometer. The protein concentration was then calculated using the extinction coefficient.

Approximately 15 mg of TCR β chain and 15 mg of TCR α chain solubilised inclusion bodies were thawed from frozen stocks and diluted into 10 ml of a guanidine solution (6 M Guanidine-hydrochloride, 50 mM Tris HCl pH 8.1, 100 mM NaCl, 10 mM EDTA, 10 mM DTT), to ensure complete chain denaturation. The guanidine solution containing fully reduced and denatured TCR chains was then injected into 0.5 litre of the following refolding buffer: 100 mM Tris pH 8.1, 400 mM L-Arginine, 2 mM EDTA, 5 M Urea. The redox couple (cysteamine hydrochloride and cystamine dihydrochloride) to final concentrations of 6.6 mM and 3.7 mM respectively, were added approximately 5 minutes before addition of the denatured TCR chains. The solution was left for ˜30 minutes. The refolded TCR was dialysed in Spectra/Por® 1 membrane (Spectrum; Product No. 132670) against 10 L H₂O for 18-20 hours. After this time, the dialysis buffer was changed twice to fresh 10 mM Tris pH 8.1 (10 L) and dialysis was continued at 5° C.±3° C. for another ˜8 hours.

Soluble TCR was separated from degradation products and impurities by loading the dialysed refold onto a POROS® 50HQ anion exchange column and eluting bound protein with a gradient of 0-500 mM NaCl in 10 mM Tris pH 8.1 over 50 column volumes using an Akta® purifier (GE Healthcare). Peak fractions were pooled and a cocktail of protease inhibitors (Calbiochem) were added. The pooled fractions were then stored at 4° C. and analysed by Coomassie-stained SDS-PAGE before being pooled and concentrated. Finally, the soluble TCR was purified and characterised using a GE Healthcare Superdex® 75HR gel filtration column pre-equilibrated in PBS buffer (Sigma). The peak eluting at a relative molecular weight of approximately 50 kDa was pooled and concentrated prior to characterisation by BIAcore® surface plasmon resonance analysis.

### Example 3

### Binding Characterisation

BIAcore Analysis

A surface plasmon resonance biosensor (BIAcore 3000®) can be used to analyse the binding of a soluble TCR to its peptide-MHC ligand. This is facilitated by producing soluble biotinylated peptide-HLA (“pHLA”) complexes which can be immobilised to a streptavidin-coated binding surface (sensor chip). The sensor chips comprise four individual flow cells which enable simultaneous measurement of T-cell receptor binding to four different pHLA complexes. Manual injection of pHLA complex allows the precise level of immobilised class I molecules to be manipulated easily.

Biotinylated class I HLA-A*02 molecules were refolded in vitro from bacterially-expressed inclusion bodies containing the constituent subunit proteins and synthetic peptide, followed by purification and in vitro enzymatic biotinylation (O'Callaghan et al. (1999) Anal. Biochem. 266: 9-15). HLA-A*02-heavy chain was expressed with a C-terminal biotinylation tag which replaces the transmembrane and cytoplasmic domains of the protein in an appropriate construct. Inclusion body expression levels of ˜75 mg/litre bacterial culture were obtained. The MHC light-chain or β2-microglobulin was also expressed as inclusion bodies in E. coli from an appropriate construct, at a level of ˜500 mg/litre bacterial culture.

E. coli cells were lysed and inclusion bodies were purified to approximately 80% purity. Protein from inclusion bodies was denatured in 6 M guanidine-HCl, 50 mM Tris pH 8.1, 100 mM NaCl, 10 mM DTT, 10 mM EDTA, and was refolded at a concentration of 30 mg/litre heavy chain, 30 mg/litre β2m into 0.4 M L-Arginine, 100 mM Tris pH 8.1, 3.7 mM cystamine dihydrochloride, 6.6 mM cysteamine hydrochloride, 4 mg/L of the EBV LMP2A peptide required to be loaded by the HLA-A*02 molecule, by addition of a single pulse of denatured protein into refold buffer at <5° C. Refolding was allowed to reach completion at 4° C. for at least 1 hour.

Buffer was exchanged by dialysis in 10 volumes of 10 mM Tris pH 8.1. The protein solution was then filtered through a 1.5 μm cellulose acetate filter and loaded onto a POROS® 50HQ anion exchange column (8 ml bed volume). Protein was eluted with a linear 0-500 mM NaCl gradient in 10 mM Tris pH 8.1 using an Akta® purifier (GE Healthcare). HLA-A*02-peptide complex eluted at approximately 250 mM NaCl, and peak fractions were collected, a cocktail of protease inhibitors (Calbiochem) was added and the fractions were chilled on ice.

Biotinylation tagged pHLA molecules were buffer exchanged into 10 mM Tris pH 8.1, 5 mM NaCl using a GE Healthcare fast desalting column equilibrated in the same buffer. Immediately upon elution, the protein-containing fractions were chilled on ice and protease inhibitor cocktail (Calbiochem) was added. Biotinylation reagents were then added: 1 mM biotin, 5 mM ATP (buffered to pH 8), 7.5 mM MgCl₂, and 5 μg/ml BirA enzyme (purified according to O'Callaghan et al. (1999) Anal. Biochem. 266: 9-15). The mixture was then allowed to incubate at room temperature overnight.

The biotinylated pHLA-A*01 molecules were purified using gel filtration chromatography. A GE Healthcare Superdex® 75 HR 10/30 column was pre-equilibrated with filtered PBS and 1 ml of the biotinylation reaction mixture was loaded and the column was developed with PBS at 0.5 ml/min using an Akta® purifier (GE Healthcare). Biotinylated pHLA-A*02 molecules eluted as a single peak at approximately 15 ml. Fractions containing protein were pooled, chilled on ice, and protease inhibitor cocktail was added. Protein concentration was determined using a Coomassie-binding assay (PerBio) and aliquots of biotinylated pHLA-A*02 molecules were stored frozen at −20° C.

The BIAcore® 3000 surface plasmon resonance (SPR) biosensor measures changes in refractive index expressed in response units (RU) near a sensor surface within a small flow cell, a principle that can be used to detect receptor ligand interactions and to analyse their affinity and kinetic parameters. The BIAcore® experiments were performed at a temperature of 25° C., using PBS buffer (Sigma, pH 7.1-7.5) as the running buffer and in preparing dilutions of protein samples. Streptavidin was immobilised to the flow cells by standard amine coupling methods. The pHLA complexes were immobilised via the biotin tag. The assay was then performed by passing soluble TCR over the surfaces of the different flow cells at a constant flow rate, measuring the SPR response in doing so.

Equilibrium Binding Constant

The above BIAcore® analysis methods were used to determine equilibrium binding constants. Serial dilutions of the disulfide linked soluble heterodimeric form of the reference EBV LMP2A TCR were prepared and injected at constant flow rate of 5 μl min⁻¹ over two different flow cells; one coated with ˜1000 RU of specific HLA-A*02 complex, the second coated with ˜1000 RU of non-specific HLA-A2-peptide complex. Response was normalised for each concentration using the measurement from the control cell. Normalised data response was plotted versus concentration of TCR sample and fitted to a non-linear curve fitting model in order to calculate the equilibrium binding constant, K_{D} (Price & Dwek, Principles and Problems in Physical Chemistry for Biochemists (2^{nd} Edition) 1979, Clarendon Press, Oxford).

Kinetic Parameters

The above BIAcore® analysis methods were also used to determine equilibrium binding constants and off-rates.

For high affinity TCRs (see Example 4 below) K_{D} was determined by experimentally measuring the dissociation rate constant, k_{off}, and the association rate constant, kₒₙ. The equilibrium constant K_{D} was calculated as k_{off}/kₒₙ.

TCR was injected over two different cells one coated with ˜1000 RU of specific EVDPIGHLY HLA-A*01 complex (“EVDPIGHLY” disclosed as SEQ ID NO: 32), the second coated with ˜1001 RU of non-specific HLA-A1-peptide complex. Flow rate was set at 50 μl/min. Typically 250 μl of TCR at ˜1 μM concentration was injected. Buffer was then flowed over until the response had returned to baseline or >2 hours had elapsed. Kinetic parameters were calculated using BIAevaluation® software. The dissociation phase was fitted to a single exponential decay equation enabling calculation of half-life.

### Example 4

### Preparation of TCRs of the Invention

Expression plasmids containing the TCR α-chain and β-chain respectively for the following TCRs of the invention were prepared as in Example 1:

| TCR ID | Alpha Chain SEQ ID No | Beta Chain SEQ ID No |
|---|---|---|
| a12b1 | 6 | 13 |
| a29b1 | 7 | 13 |
| a32b1 | 8 | 13 |
| a37b1 | 9 | 13 |
| a38b1 | 10 | 13 |
| a42b1-antiCD3* | 11 | 16 |
| a43b1-antiCD3* | 12 | 16 |

*prepared as described in example 5

The plasmids were transformed separately into E. coli strain BL21pLysS, and single ampicillin-resistant colonies grown at 37° C. in TYP (ampicillin 100 μg/ml) medium to OD₆₀₀ of ˜0.6-0.8 before inducing protein expression with 0.5 mM IPTG. Cells were harvested three hours post-induction by centrifugation for 30 minutes at 4000 rpm in a Beckman J-6B. Cell pellets were lysed with 25 ml Bug Buster (Novagen) in the presence of MgCl₂ and DNaseI. Inclusion body pellets were recovered by centrifugation for 30 minutes at 13000 rpm in a Beckman J2-21 centrifuge. Three detergent washes were then carried out to remove cell debris and membrane components. Each time the inclusion body pellet was homogenised in a Triton buffer (50 mM Tris-HCl pH 8.0, 0.5% Triton-X100, 200 mM NaCl, 10 mM NaEDTA,) before being pelleted by centrifugation for 15 minutes at 13000 rpm in a Beckman J2-21. Detergent and salt was then removed by a similar wash in the following buffer: 50 mM Tris-HCl pH 8.0, 1 mM NaEDTA. Finally, the inclusion bodies were divided into 30 mg aliquots and frozen at −70° C. Inclusion body protein yield was quantified by solubilising with 6 M guanidine-HCl and an OD measurement was taken on a Hitachi U-2001 Spectrophotometer. The protein concentration was then calculated using the extinction coefficient.

Approximately 10 mg of TCR β chain and 10 mg of TCR α chain solubilised inclusion bodies for each TCR of the invention were diluted into 10 ml of a guanidine solution (6 M Guanidine-hydrochloride, 50 mM Tris HCl pH 8.1, 100 mM NaCl, 10 mM EDTA, 10 mM DTT), to ensure complete chain denaturation. The guanidine solution containing fully reduced and denatured TCR chains was then injected into 0.5 litre of the following refolding buffer: 100 mM Tris pH 8.1, 400 mM L-Arginine, 2 mM EDTA, 5 M Urea. The redox couple (cysteamine hydrochloride and cystamine dihydrochloride) to final concentrations of 6.6 mM and 3.7 mM respectively, were added approximately 5 minutes before addition of the denatured TCR chains. The solution was left for ˜30 minutes. The refolded TCR was dialysed in Spectra/Por® 1 membrane (Spectrum; Product No. 132670) against 10 L H₂O for 18-20 hours. After this time, the dialysis buffer was changed twice to fresh 10 mM Tris pH 8.1 (10 L) and dialysis was continued at 5° C.±3° C. for another ˜8 hours.

Soluble TCR was separated from degradation products and impurities by loading the dialysed refold onto a POROS® 50HQ anion exchange column and eluting bound protein with a gradient of 0-500 mM NaCl in 10 mM Tris pH 8.1 over 15 column volumes using an Akta® purifier (GE Healthcare). The pooled fractions were then stored at 4° C. and analysed by Coomassie-stained SDS-PAGE before being pooled and concentrated. Finally, the soluble TCRs were purified and characterised using a GE Healthcare Superdex® 75HR gel filtration column pre-equilibrated in PBS buffer (Sigma). The peak eluting at a relative molecular weight of approximately 50 kDa was pooled and concentrated prior to characterisation by BIAcore® surface plasmon resonance analysis.

The affinity profiles of the thus-prepared TCRs for the EBV LMP2A epitope were assessed using the method of Example 3, and compared with the reference TCR. The results are set forth in the following table:

| | | |
|---|---|---|
| TCR ID | T½ | K_{D} |

| | | | | |
|---|---|---|---|---|
| Reference | 3.45 | s | 23 | μM |
| a12b1 | 190.6 | min | 294 | pM |
| a29b1 | 10.2 | h | 55 | pM |
| a32b1 | 25 | h | 52 | pM |
| a37b1 | 8 | h | 44 | pM |
| a38b1 | 16 | h | 144 | pM |
| a42b1-antiCD3 | 60 | h | 14 | pM |
| a43b1-antiCD3 | 20 | h | 23 | pM |

### Example 5

### Expression, Refolding and Purification of Soluble Anti-CD3 scFv-EBV LMP2A TCR Fusion

TCRs comprising an alpha chain and an anti-CD3 scFv-TCR beta chain fusion were produced as described below.

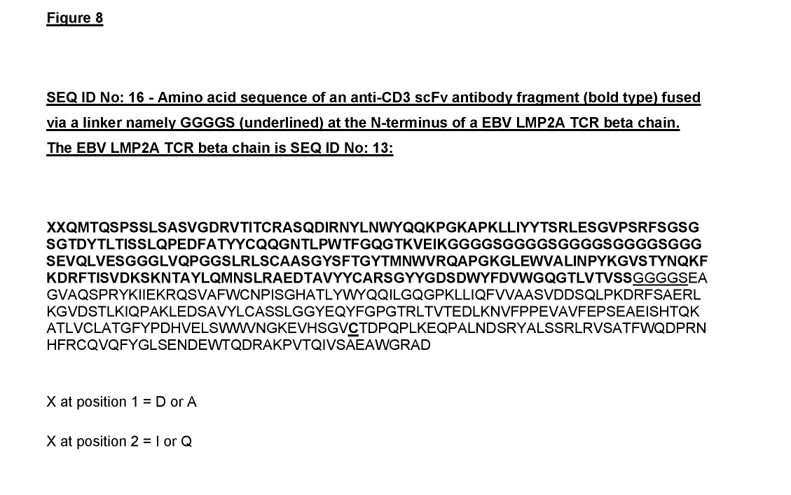

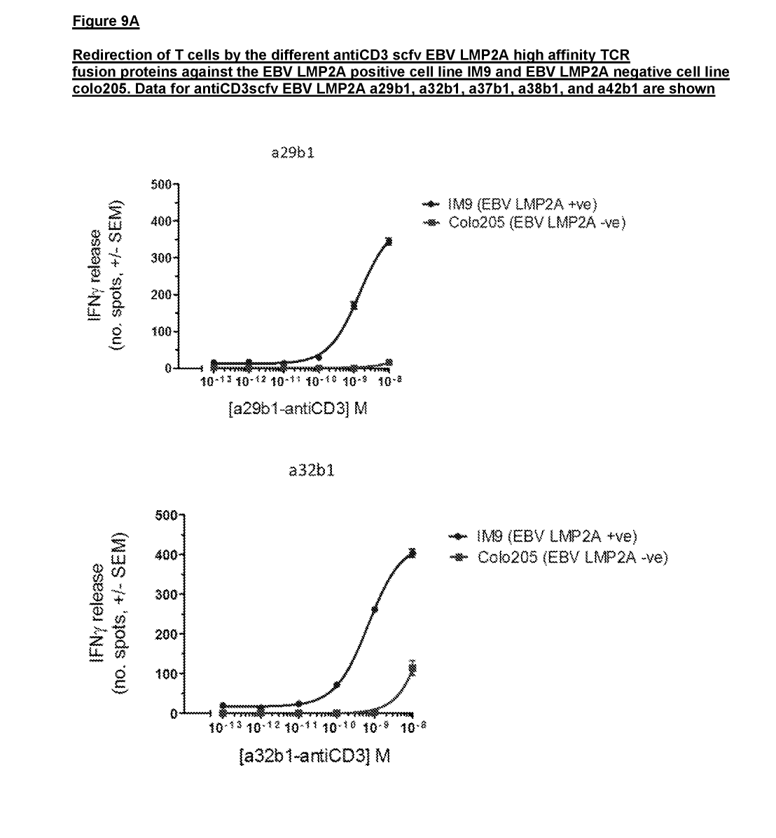

Alpha chain sequences were selected from SEQ ID No: 6, SEQ ID No: 7, SEQ ID No: 8, SEQ ID No: 9, SEQ ID No: 10, SEQ ID No: 11, or SEQ ID No: 12. The beta chain sequence was SEQ ID No:13. This sequence was fused to an anti-CD3 scFV sequence via a linker. The linker was selected from the group consisting of GGGGS (SEQ ID No: 23), GGGSG (SEQ ID No: 24), GGSGG (SEQ ID No: 25), GSGGG (SEQ ID No: 26), GSGGGP (SEQ ID No: 27), GGEPS (SEQ ID No: 28), GGEGGGP (SEQ ID No: 29) and GGEGGGSEGGGS (SEQ ID No: 30). The amino acid at position 1 of the anti-CD3 scFV sequence (shown in FIG. 8) was either D or A and the amino acid at position 2 was either I or Q.

The constructs were prepared as follows:

Ligation

Synthetic genes encoding (a) the TCR Vα chain and (b) the TCR Vβ chain fusion sequence described above, were separately ligated into pGMT7+Cα vector and pGMT7-based expression plasmid respectively, which contain the T7 promoter for high level expression in E. coli strain BL21-DE3(pLysS) (Pan et al., Biotechniques (2000) 29 (6): 1234-8).

Expression

The expression plasmids were transformed separately into E. coli strain BL21 (DE3) Rosetta pLysS, and single ampicillin-resistant colonies were grown at 37° C. in TYP (ampicillin 100 μg/ml) medium to OD₆₀₀ of ˜0.6-0.8 before inducing protein expression with 0.5 mM IPTG. Cells were harvested three hours post-induction by centrifugation for 30 minutes at 4000 rpm in a Beckman J-6B. Cell pellets were lysed with 25 ml Bug Buster® (NovaGen) in the presence of MgCl₂ and DNase. Inclusion body pellets were recovered by centrifugation for 30 minutes at 13000 rpm in a Beckman J2-21 centrifuge. Three detergent washes were then carried out to remove cell debris and membrane components. Each time the inclusion body pellet was homogenised in a Triton buffer (50 mM Tris-HCl pH 8.0, 0.5% Triton-X100, 200 mM NaCl, 10 mM NaEDTA,) before being pelleted by centrifugation for 15 minutes at 13000 rpm in a Beckman J2-21. Detergent and salt was then removed by a similar wash in the following buffer: 50 mM Tris-HCl pH 8.0, 1 mM NaEDTA. Finally, the inclusion bodies were divided into 30 mg aliquots and frozen at −70° C.

Refolding

Approximately 20 mg of TCR α chain and 40 mg of scFv-TCR β chain solubilised inclusion bodies were thawed from frozen stocks, diluted into 20 ml of a guanidine solution (6 M Guanidine-hydrochloride, 50 mM Tris HCl pH 8.1, 100 m NaCl, 10 mM EDTA, 10 mM DTT), and incubated in a 37° C. water bath for 30 min-1 hr to ensure complete chain de-naturation. The guanidine solution containing fully reduced and denatured TCR chains was then injected into 1 litre of the following refolding buffer: 100 mM Tris pH 8.1, 400 mM L-Arginine, 2 mM EDTA, 5M Urea. The redox couple (cysteamine hydrochloride and cystamine dihydrochloride (to final concentrations of 10 mM and 2.5 mM, respectively)) were added approximately 5 minutes before addition of the denatured TCR α and scFv-TCR β chains. The solution was left for ˜30 minutes. The refolded scFv-TCR was dialysed in dialysis tubing cellulose membrane (Sigma-Aldrich; Product No. D9402) against 10 L H₂O for 18-20 hours. After this time, the dialysis buffer was changed twice to fresh 10 mM Tris pH 8.1 (10 L) and dialysis was continued at 5° C.±3° C. for another ˜8 hours. Soluble and correctly folded scFv-TCR was separated from degradation products and impurities by a 3-step purification method as described below.

First Purification Step

The dialysed refold (in 10 mM Tris pH8.1) was loaded onto a POROS® 50HQ anion exchange column and the bound protein eluted with a gradient of 0-500 mM NaCl over 6 column volumes using an Akta purifier (GE Healthcare). Peak fractions (eluting at a conductivity ˜20 mS/cm) were stored at 4° C. Peak fractions were analysed by Instant Blue® Stain (Novexin) stained SDS-PAGE before being pooled.

Second Purification Step

The anion exchange pooled fractions were buffer exchanged by dilution with 20 mM IVIES pH6-6.5, depending on the pI of the scFv-TCR fusion. The soluble and correctly folded scFv-TCR was separated from degradation products and impurities by loading the diluted pooled fractions (in 20 mM IVIES pH6-6.5) onto a POROS® 50HS cation exchange column and eluting bound protein with a gradient of 0-500 mM NaCl over 6 column volumes using an Akta® purifier (GE Healthcare). Peak fractions (eluting at a conductivity ˜10 mS/cm) were stored at 4° C.

Final Purification Step

Peak fractions from second purification step were analysed by Instant Blue® Stain (Novexin) stained SDS-PAGE before being pooled. The pooled fractions were then concentrated for the final purification step, when the soluble scFv-TCR was purified and characterised using a Superdex® S200 gel filtration column (GE Healthcare) pre-equilibrated in PBS buffer (Sigma). The peak eluting at a relative molecular weight of approximately 78 kDa was analysed by Instant Blue® Stain (Novexin) stained SDS-PAGE before being pooled.

### Example 6

### Redirection of T Cells by Anti-CD3 scFv-EBV LMP2A High Affinity TCR Fusions Against Peptide Pulsed T2 Cells and Three Cell Lines; IM9, HCT116 and Colo205

Five anti-CD3 scFv-EBV LMP2A TCR fusions were produced as described in Example 5. The sequences of the alpha and beta chains were as follows:

| TCR ID | Alpha Chain SEQ ID No | Beta Chain SEQ ID No |
|---|---|---|
| a29b1-antiCD3 | 7 | 13 |
| a32b1-antiCD3 | 8 | 13 |
| a37b1-antiCD3 | 9 | 13 |
| a38b1-antiCD3 | 10 | 13 |
| a42b1-antiCD3 | 11 | 13 |

The following assays were carried out to demonstrate activation of cytotoxic T lymphocytes (CTLs) by anti-CD3 scFv-EBV LMP2A TCR fusions via specific peptide-MHC recognition. IFN-γ production, measured by the ELISPOT assay, was used as a read-out for CTL activation and enabled evaluation of the potency of the anti-CD3 scFv portion of the fusion proteins.

Assay media: 10% FCS (Heat Inactivated, Sera Laboratories international, cat# EU-000-F1), 88% RPMI 1640 (Invitrogen, cat#42401018), 1% glutamine (Invitrogen, cat#25030024) and 1% penicillin/streptomycin (lnvitrogen, cat#15070063).

Wash buffer: 1×PBS sachet (Sigma, cat# P3813), containing 0.05% Tween-20, made up in deionised water

PBS (lnvitrogen, cat#10010015)

Dilution Buffer: PBS and 10% FCS (Heat Inactivated)

The Human IFNγ ELISPOT PVDF-Enzymatic kit (BD Biosciences, cat#551849) contains all other reagents required (capture and detection antibodies, streptavidin-HRP and substrate solution as the Human IFN-γ PVDF ELISPOT 96 well plates)

Method

Target Cell Preparation

Target cells were characterised for EBV LMP2A antigen expression by quantitative RT-PCR using standard procedures and primers specific for the antigen. The IM9 target cells used in the assay were shown to express EBV LMP2A; colo205 colorectal cell-line is EBV LMP2A-ve. Sufficient target cells (to allow for 50,000 cells/well in the assay) were washed by centrifugation three times at 1200 rpm for 5 min in a Megafuge 1.0 (Heraeus). Cells were then re-suspended in assay media at a density of 10⁶ cells/ml.

Effector Cell Preparation

The effector cells (T cells) used in this method were peripheral blood mononuclear cells (PBMC). PBMCs were isolated from blood using standard procedures utilising Lymphoprep (Axis-Shields, cat# NYC-1114547) and Leucosep tubes (Greiner, cat#227290). Effector cells were defrosted and placed in assay media prior to washing by centrifugation at 1300 rpm for 10 min in a Megafuge 1.0 (Heraeus). Cells were then re-suspended in assay media at 4× the final required concentration.

Reagent/Test Compound Preparation

Varying concentrations of the anti-CD3 scFv-EBV LMP2A TCR fusion proteins (from 10 nM to 0.1 pM) were prepared by dilution into assay media to give 4× the final concentration.

ELISPOTs

Plates were prepared as follows: 100 μl anti-IFN-γ or anti-GrB capture antibody was diluted in 10 ml sterile PBS per plate. 100 μl of the diluted capture antibody was then dispensed into each well. The plates were then incubated overnight at 4° C. Following incubation the plates were washed (programme 1, plate type 2, Ultrawash Plus 96-well plate washer; Dynex) to remove the capture antibody. Plates were then blocked by adding 200 μl of assay media to each well and incubated at room temperature for two hours. The assay media was then washed from the plates (programme 1, plate type 2, Ultrawash Plus 96-well plate washer, Dynex) and any remaining media was removed by flicking and tapping the ELISPOT plates on a paper towel.

The constituents of the assay were then added to the ELISPOT plate in the following order:
50 μl of target cells 10⁶ cells/ml (giving a total of 50,000 target cells/well)
50 μl of reagent (the anti-CD3 scFv-TCR fusions; varying concentrations)
50 μl media (assay media)
50 μl effector cells (20,000 PBMC cells/well)

The plates were then incubated overnight (37° C./5% CO₂). The next day the plates were washed three times (programme 1, plate type 2, Ultrawash Plus 96-well plate washer, Dynex) with wash buffer and tapped dry on paper towel to remove excess wash buffer. 100 μl of primary detection antibody was then added to each well. The primary detection antibody was diluted into 10 ml of dilution buffer (the volume required for a single plate) using the dilution specified in the manufacturer's instructions. Plates were then incubated at room temperature for at least 2 hours prior to being washed three times (programme 1, plate type 2, Ultrawash Plus 96-well plate washer, Dynex) with wash buffer; excess wash buffer was removed by tapping the plate on a paper towel.

Secondary detection was performed by adding 100 μl of diluted streptavidin-HRP to each well and incubating the plate at room temperature for 1 hour. The streptavidin-HRP was diluted into 10 ml dilution buffer (the volume required for a single plate), using the dilution specified in the manufacturer's instructions. The plates were then washed three times (programme 1, plate type 2, Ultrawash Plus 96-well plate washer, Dynex) with wash buffer and tapped on paper towel to remove excess wash buffer. Plates were then washed twice with PBS by adding 200 μl to each well, flicking the buffer off and tapping on a paper towel to remove excess buffer. No more than 15 mins prior to use, one drop (20 μl) of AEC chromogen was added to each 1 ml of AEC substrate and mixed. 10 ml of this solution was prepared for each plate; 100 μl was added per well. The plate was then protected from light using foil, and spot development monitored regularly, usually occurring within 5-20 mins. The plates were washed in tap water to terminate the development reaction, and shaken dry prior to their disassembly into three constituent parts. The plates were then allowed to dry at room temperature for at least 2 hours prior to counting the spots using an Immunospot Plate reader (CTL; Cellular Technology Limited).

Results

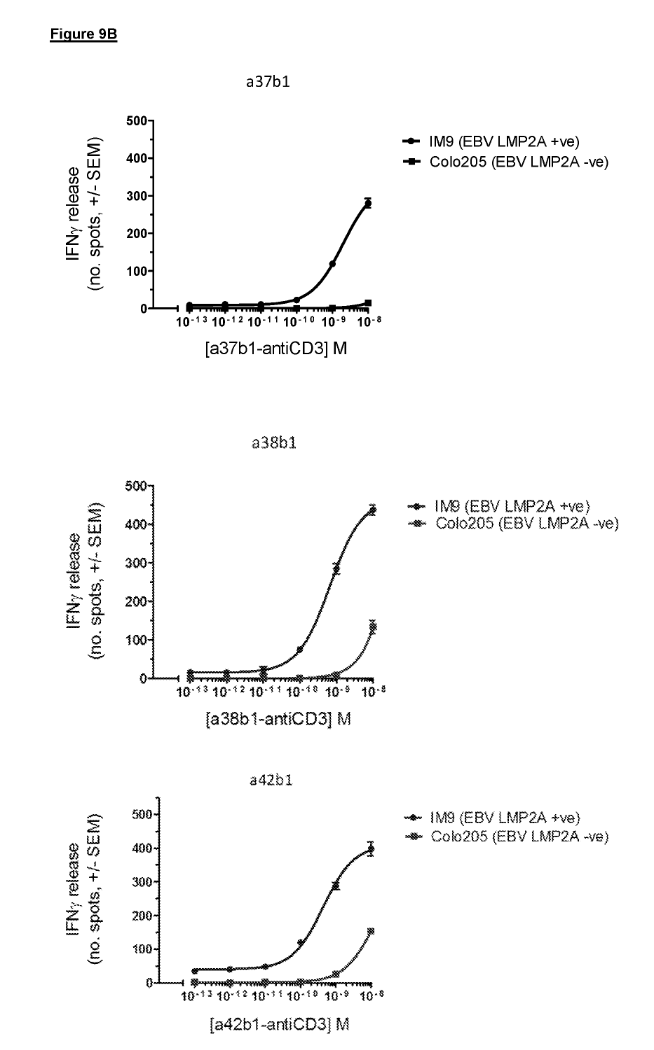

The anti-CD3 scFv-EBV LMP2A TCR fusions were tested by ELISPOT Assay (as described above). The number of spots observed in each well was plotted against the concentration of the fusion construct using Prism (Graph Pad) (see FIG. 9). From these dose-response curves, the EC₅₀ values were determined (EC₅₀ are determined at the concentration of anti-CD3 scFv-EBV LMP2A TCR fusion that induces 50% of the maximum response).

The graphs in FIG. 9 show the specific activation of T cells by the different anti-CD3 scFv-EBV LMP2A high affinity TCRs in the presence of EBV LMP2A presenting cell line IM9. The data is representative of at least three separate assays in each case.

The data from FIG. 9 yields the following EC₅₀ values; 1430 pM for a29b1-antiCD3, 681 pM for a32b1-antiCD3, 1980 pM for a37b1-antiCD3, 66.7 pM for a38b-antiCD3 and 439 pM for a42b1-antiCD3.

The invention is further described by the following numbered paragraphs:

1. A T cell receptor (TCR) having the property of binding to CLGGLLTMV (SEQ ID No: 1) HLA-A2 complex and comprising a TCR alpha chain variable domain and/or a TCR beta chain variable domain,
the alpha chain variable domain comprising an amino acid sequence that has at least 80% identity to the sequence of amino acid residues 1-113 of SEQ ID No: 2, and/or
the beta chain variable domain comprising an amino acid sequence that has at least 80% identity to the sequence of amino acid residues 1-112 of SEQ ID No: 3,
wherein the alpha chain variable domain has at least one of the following mutations:

| Residue no. | | |
|---|---|---|
| 28 | P | |
| 29 | H | |
| 30 | M | |
| 31 | A | |
| 50 | L | Q |
| 51 | P | F |
| 52 | G | Q |
| 53 | G | D |
| 94 | D | |
| 96 | Y | H |
| 97 | G | Q |
| 98 | H | P |
| 100 | R | |

and/or the beta chain variable domain has at least one of the following mutations:

| Residue no. | |
|---|---|
| 50 | V |
| 51 | V |
| 52 | A |
| 53 | A |
| 54 | S |

2. The TCR of paragraph 1, wherein (i) the alpha chain variable domain comprises Q1 to P113 of SEQ ID No: 6; Q1 to P113 SEQ ID No: 7, Q1 to P113 SEQ ID No: 8, Q1 to P113 SEQ ID No: 9, Q1 to P113 SEQ ID No: 10, Q1 to P113 SEQ ID No: 11 or Q1 to P113 SEQ ID No: 12, and (ii) the beta chain comprises E1 to T112 of SEQ ID No: 13.

3. The TCR of paragraph 1 or paragraph 2, which is an alpha-beta heterodimer, having an alpha chain TRAC constant domain sequence and/or a beta chain TRBC1 or TRBC2 constant domain sequence.

4. The TCR of paragraph 3, wherein the alpha and beta chain constant domain sequences are modified by truncation or substitution to delete the native disulfide bond between Cys4 of exon 2 of TRAC and Cys2 of exon 2 of TRBC1 or TRBC2.

5. The TCR of paragraph 3 or paragraph 4 wherein the alpha and/or beta chain constant domain sequence(s) are modified by substitution of cysteine residues for Thr 48 of TRAC and Ser 57 of TRBC1 or TRBC2, the said cysteines forming a disulfide bond between the alpha and beta constant domains of the TCR.

6. The TCR of paragraph 1 or paragraph 2, which is in single chain format of the type Vα-L-Vβ, Vβ-L-Vα, Vα-Cα-L-Vβ, Vα-:-Vβ-Cβ, wherein Va and VP are TCR α and β variable regions respectively, Cα and Cβ are TCR α and β constant regions respectively, and L is a linker sequence.

7. The TCR of any preceding paragraph associated with a detectable label, a therapeutic agent or a PK modifying moiety.

8. The TCR of any preceding paragraph, comprising an anti-CD3 antibody covalently linked to the C- or N-terminus of the alpha or beta chain of the TCR.

9. The TCR of paragraph 8, which has the alpha chain SEQ ID No: 6, 7, 8, 9, 10, 11 or 12 and the beta chain SEQ ID No: 13 fused to an anti-CD3 antibody.

10. The TCR of paragraph 9, wherein the beta chain is linked to the anti-CD3 antibody sequence via a linker sequence.

11. The TCR of paragraph 10, wherein the linker sequence is selected from the group consisting of GGGGS (SEQ ID No: 23), GGGSG (SEQ ID No: 24), GGSGG (SEQ ID No: 25), GSGGG (SEQ ID No: 26), GSGGGP (SEQ ID No: 27), GGEPS (SEQ ID No: 28), GGEGGGP (SEQ ID No: 29), and GGEGGGSEGGGS (SEQ ID No: 30).

12. The TCR of paragraph 8, comprising (a) an alpha chain amino acid sequence and (b) a beta chain-anti-CD3 amino acid sequence, wherein the alpha chain sequence selected from the group consisting of:
(i) the TCR alpha chain sequence SEQ ID No: 2 or SEQ ID No: 4, wherein amino acids 1 to 113 are replaced by the amino acids 1-113 of sequence SEQ ID No: 9;
(ii) the TCR alpha chain sequence SEQ ID No: 2 or SEQ ID No: 4, wherein amino acids 1 to 113 are replaced by the amino acids 1-113 of sequence SEQ ID No: 9, wherein the amino acid at position 1 is replaced by A;
(iii) the TCR alpha chain sequence SEQ ID No: 2 or SEQ ID No: 4, wherein amino acids 1 to 113 are replaced by the amino acids 1-113 of sequence SEQ ID No: 9, wherein the amino acid at position 1 is replaced by G;
(iv) the TCR alpha chain sequence SEQ ID No: 2 or SEQ ID No: 4, wherein amino acids 1 to 113 are replaced by the amino acids 1-113 of sequence SEQ ID No: 9, and the C-terminus of the alpha chain is truncated by 8 amino acids from F200 to S207 inclusive, based on the numbering of SEQ ID No: 4;
(v) the TCR alpha chain sequence SEQ ID No: 2 or SEQ ID No: 4, wherein amino acids 1 to 113 are replaced by the amino acids 1-113 of sequence SEQ ID No: 9, wherein the amino acid at position 1 is replaced by A, and the C-terminus of the alpha chain is truncated by 8 amino acids from F200 to S207 inclusive, based on the numbering of SEQ ID No: 4;
(vi) the TCR alpha chain sequence SEQ ID No: 2 or SEQ ID No: 4, wherein amino acids 1 to 113 are replaced by the amino acids 1-113 of sequence SEQ ID No: 9, wherein the amino acid at position 1 is replaced by G, and the C-terminus of the alpha chain is truncated by 8 amino acids from F200 to S207 inclusive, based on the numbering of SEQ ID No: 4;
   and/or the beta chain-anti-CD3 amino acid sequence is selected from the group consisting of:
(vii) the TCR beta chain-anti-CD3 sequence SEQ ID No: 16, wherein amino acids at positions 1 and 2 are D and I respectively;
(viii) the TCR beta chain-anti-CD3 sequence SEQ ID No: 16, wherein amino acids at positions 1 and 2 are A and I respectively;
(ix) the TCR beta chain-anti-CD3 sequence SEQ ID No: 16, wherein amino acids at positions 1 and 2 are A and Q respectively;
(x) the TCR beta chain-anti-CD3 sequence SEQ ID No: 16, wherein amino acids at positions 1 and 2 are D and I respectively, amino acids at positions 108-131 are replaced by RTSGPGDGGKGGPGKGPGGEGTKGTGPGG (SEQ ID No: 31), and amino acids at positions 254-258 are replaced by GGEGGGSEGGGS (SEQ ID No: 30);
(xi) the TCR beta chain-anti-CD3 sequence SEQ ID No: 16, wherein amino acids at positions 1 and 2 are D and I respectively, the amino acid at position 257 is S and the amino acid at position 258 is G;
(xii) the TCR beta chain-anti-CD3 sequence SEQ ID No: 16, wherein amino acids at positions 1 and 2 are D and I respectively, the amino acid at position 256 is S and the amino acid at position 258 is G;
(xiii) the TCR beta chain-anti-CD3 sequence SEQ ID No: 16, wherein amino acids at positions 1 and 2 are D and I respectively, the amino acid at position 255 is S and the amino acid at position 258 is G;
(xiv) a TCR beta chain-anti-CD3 having the sequence SEQ ID No: 16, wherein amino acids at positions 1 and 2 are A and Q, the amino acid at position 257 is S and the amino acid at position 258 is G.
(xv) a TCR beta chain-anti-CD3 having the sequence SEQ ID No: 16, wherein amino acids at positions 1 and 2 are A and Q, the amino acid at position 256 is S and the amino acid at position 258 is G;
(xvi) a TCR beta chain-anti-CD3 having the sequence SEQ ID No: 16, wherein amino acids at positions 1 and 2 are A and Q, the amino acid at position 255 is S and the amino acid at position 258 is G;
(xvii) and a TCR beta chain-anti-CD3 having the sequence SEQ ID No: 16, wherein amino acid at positions 1 and 2 are A and I respectively, the amino acid at position 257 is S and the amino acid at position 258 is G;
(xviii) a TCR beta chain-anti-CD3 having the sequence SEQ ID No: 16, wherein amino acid at positions 1 and 2 are A and I respectively, the amino acid at position 256 is S and the amino acid at position 258 is G;
(xix) a TCR beta chain-anti-CD3 having the sequence SEQ ID No: 16, wherein amino acid at positions 1 and 2 are A and I respectively, the amino acid at position 255 is S and the amino acid at position 258 is G.

13. The TCR of paragraph 12, wherein the combination of alpha chain sequence and beta chain-anti-CD3 sequence is selected from:
the alpha chain amino acid sequence is (i) and the beta chain-anti-CD3 amino acid sequence is (vii);
the alpha chain amino acid sequence is (i) and the beta chain-anti-CD3 amino acid sequence is (x);
the alpha chain amino acid sequence is (vi) and the beta chain-anti-CD3 amino acid sequence is (ix);
the alpha chain amino acid sequence is (v) and the beta chain-anti-CD3 amino acid sequence is (viii);
the alpha chain amino acid sequence is (vi) and the beta chain-anti-CD3 amino acid sequence is (vii);
the alpha chain amino acid sequence is (i) and the beta chain-anti-CD3 amino acid sequence is (xi);
the alpha chain amino acid sequence is (i) and the beta chain-anti-CD3 amino acid sequence is (xii);
the alpha chain amino acid sequence is (i) and the beta chain-anti-CD3 amino acid sequence is (xiii);
the alpha chain amino acid sequence is (vi) and the beta chain-anti-CD3 amino acid sequence is (xiv);
the alpha chain amino acid sequence is (vi) and the beta chain-anti-CD3 amino acid sequence is (xv);
the alpha chain amino acid sequence is (vi) and the beta chain-anti-CD3 amino acid sequence is (xvi);
the alpha chain amino acid sequence is (v) and the beta chain-anti-CD3 amino acid sequence is (xvii);
the alpha chain amino acid sequence is (v) and the beta chain-anti-CD3 amino acid sequence is (xviii);
the alpha chain amino acid sequence is (v) and the beta chain-anti-CD3 amino acid sequence is (xix);
the alpha chain amino acid sequence is (vi) and the beta chain-anti-CD3 amino acid sequence is (xi);
the alpha chain amino acid sequence is (vi) and the beta chain-anti-CD3 amino acid sequence is (xii);
the alpha chain amino acid sequence is (vi) and the beta chain-anti-CD3 amino acid sequence is (xiii).

14. Nucleic acid encoding a TCR of any one of the preceding paragraphs.

15. A non-naturally occurring and/or purified and/or engineered cell, especially a T-cell, presenting a TCR of any one of paragraphs 1 to 11.

16. A cell harbouring
(a) a TCR expression vector which comprises nucleic acid of paragraph 14 in a single open reading frame, or two distinct open reading frames encoding the alpha chain and the beta chain respectively; or
(b) a first expression vector which comprises nucleic acid encoding the alpha chain of a TCR of any of paragraphs 1 to 13, and a second expression vector which comprises nucleic acid encoding the beta chain of a TCR of any of paragraphs 1 to 13.

17. A pharmaceutical composition comprising a TCR of any one of paragraphs 1 to 13 or a cell of paragraph 15 or paragraph 16, together with one or more pharmaceutically acceptable carriers or excipients.

18. A TCR which binds the CLGGLLTMV peptide (SEQ ID NO: 1) (derived from the EBV LMP2A protein) presented as a peptide-HLA-A2 complex, or a cell expressing and/or presenting such a TCR, for use in medicine.

19. The TCR or cell for use of paragraph 18, for use in a method of treating EBV infection.

20. The TCR or cell for use of paragraph 18 or paragraph 19, wherein the TCR is of any one of paragraphs 1 to 13 and/or wherein the cell is of paragraph 15 or paragraph 16.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A T cell receptor (TCR) having the property of binding to CLGGLLTMV (SEQ ID No: 1) HLA-A2 complex and comprising a TCR alpha chain variable domain and/or a TCR beta chain variable domain,
the alpha chain variable domain comprising an amino acid sequence that has at least 80% identity to the sequence of amino acid residues 1-113 of SEQ ID No: 2, and/or
the beta chain variable domain comprising an amino acid sequence that has at least 80% identity to the sequence of amino acid residues 1-112 of SEQ ID No: 3,
wherein the alpha chain variable domain has at least one of the following mutations:

| Residue no. | | |
|---|---|---|
| 28 | P | |
| 29 | H | |
| 30 | M | |
| 31 | A | |
| 50 | L | Q |
| 51 | P | F |
| 52 | G | Q |
| 53 | G | D |
| 94 | D | |
| 96 | Y | H |
| 97 | G | Q |
| 98 | H | P |
| 100 | R | |

and/or the beta chain variable domain has at least one of the following mutations:

| Residue no. | |
|---|---|
| 50 | V |
| 51 | V |
| 52 | A |
| 53 | A |
| 54 | S |

2. The TCR of claim 1, wherein (i) the alpha chain variable domain comprises Q1 to P113 of SEQ ID No: 6; Q1 to P113 SEQ ID No: 7, Q1 to P113 SEQ ID No: 8, Q1 to P113 SEQ ID No: 9, Q1 to P113 SEQ ID No: 10, Q1 to P113 SEQ ID No: 11 or Q1 to P113 SEQ ID No: 12, and (ii) the beta chain comprises E1 to T112 of SEQ ID No: 13.

3. The TCR of claim 1, which is an alpha-beta heterodimer, having an alpha chain TRAC constant domain sequence and/or a beta chain TRBC1 or TRBC2 constant domain sequence.

4. The TCR of claim 3, wherein the alpha and beta chain constant domain sequences are modified by truncation or substitution to delete the native disulfide bond between Cys4 of exon 2 of TRAC and Cys2 of exon 2 of TRBC1 or TRBC2.

5. The TCR of claim 3 wherein the alpha and/or beta chain constant domain sequence(s) are modified by substitution of cysteine residues for Thr 48 of TRAC and Ser 57 of TRBC1 or TRBC2, the said cysteines forming a disulfide bond between the alpha and beta constant domains of the TCR.

6. The TCR of claim 1, which is in single chain format of the type Vα-L-Vβ, Vβ-L-Vα, Vα-Cα-L-Vβ, Vα-:-Vβ-Cβ, wherein Vα and Vβ are TCR α and β variable regions respectively, Cα and Cβ are TCR α and β constant regions respectively, and L is a linker sequence.

7. The TCR of claim 1 associated with a detectable label, a therapeutic agent or a PK modifying moiety.

8. The TCR of claim 1, comprising an anti-CD3 antibody covalently linked to the C- or N-terminus of the alpha or beta chain of the TCR.

9. The TCR of claim 8, which has the alpha chain SEQ ID No: 6, 7, 8, 9, 10, 11 or 12 and the beta chain SEQ ID No: 13 fused to an anti-CD3 antibody.

10. The TCR of claim 9, wherein the beta chain is linked to the anti-CD3 antibody sequence via a linker sequence.

11. The TCR of claim 10, wherein the linker sequence is selected from the group consisting of GGGGS (SEQ ID No: 23), GGGSG (SEQ ID No: 24), GGSGG (SEQ ID No: 25), GSGGG (SEQ ID No: 26), GSGGGP (SEQ ID No: 27), GGEPS (SEQ ID No: 28), GGEGGGP (SEQ ID No: 29), and GGEGGGSEGGGS (SEQ ID No: 30).

12. The TCR of claim 8, comprising (a) an alpha chain amino acid sequence and (b) a beta chain-anti-CD3 amino acid sequence, wherein the alpha chain sequence selected from the group consisting of:
(i) the TCR alpha chain sequence SEQ ID No: 2 or SEQ ID No: 4, wherein amino acids 1 to 113 are replaced by the amino acids 1-113 of sequence SEQ ID No: 9;
(ii) the TCR alpha chain sequence SEQ ID No: 2 or SEQ ID No: 4, wherein amino acids 1 to 113 are replaced by the amino acids 1-113 of sequence SEQ ID No: 9, wherein the amino acid at position 1 is replaced by A;
(iii) the TCR alpha chain sequence SEQ ID No: 2 or SEQ ID No: 4, wherein amino acids 1 to 113 are replaced by the amino acids 1-113 of sequence SEQ ID No: 9, wherein the amino acid at position 1 is replaced by G;
(iv) the TCR alpha chain sequence SEQ ID No: 2 or SEQ ID No: 4, wherein amino acids 1 to 113 are replaced by the amino acids 1-113 of sequence SEQ ID No: 9, and the C-terminus of the alpha chain is truncated by 8 amino acids from F200 to S207 inclusive, based on the numbering of SEQ ID No: 4;
(v) the TCR alpha chain sequence SEQ ID No: 2 or SEQ ID No: 4, wherein amino acids 1 to 113 are replaced by the amino acids 1-113 of sequence SEQ ID No: 9, wherein the amino acid at position 1 is replaced by A, and the C-terminus of the alpha chain is truncated by 8 amino acids from F200 to S207 inclusive, based on the numbering of SEQ ID No: 4;
(vi) the TCR alpha chain sequence SEQ ID No: 2 or SEQ ID No: 4, wherein amino acids 1 to 113 are replaced by the amino acids 1-113 of sequence SEQ ID No: 9, wherein the amino acid at position 1 is replaced by G, and the C-terminus of the alpha chain is truncated by 8 amino acids from F200 to S207 inclusive, based on the numbering of SEQ ID No: 4;
and/or the beta chain-anti-CD3 amino acid sequence is selected from the group consisting of:
(vii) the TCR beta chain-anti-CD3 sequence SEQ ID No: 16, wherein amino acids at positions 1 and 2 are D and I respectively;
(viii) the TCR beta chain-anti-CD3 sequence SEQ ID No: 16, wherein amino acids at positions 1 and 2 are A and I respectively;
(ix) the TCR beta chain-anti-CD3 sequence SEQ ID No: 16, wherein amino acids at positions 1 and 2 are A and Q respectively;
(x) the TCR beta chain-anti-CD3 sequence SEQ ID No: 16, wherein amino acids at positions 1 and 2 are D and I respectively, amino acids at positions 108-131 are replaced by RTSGPGDGGKGGPGKGPGGEGTKGTGPGG (SEQ ID No: 31), and amino acids at positions 254-258 are replaced by GGEGGGSEGGGS (SEQ ID No: 30);
(xi) the TCR beta chain-anti-CD3 sequence SEQ ID No: 16, wherein amino acids at positions 1 and 2 are D and I respectively, the amino acid at position 257 is S and the amino acid at position 258 is G;
(xii) the TCR beta chain-anti-CD3 sequence SEQ ID No: 16, wherein amino acids at positions 1 and 2 are D and I respectively, the amino acid at position 256 is S and the amino acid at position 258 is G;
(xiii) the TCR beta chain-anti-CD3 sequence SEQ ID No: 16, wherein amino acids at positions 1 and 2 are D and I respectively, the amino acid at position 255 is S and the amino acid at position 258 is G;
(xiv) a TCR beta chain-anti-CD3 having the sequence SEQ ID No: 16, wherein amino acids at positions 1 and 2 are A and Q, the amino acid at position 257 is S and the amino acid at position 258 is G;
(xv) a TCR beta chain-anti-CD3 having the sequence SEQ ID No: 16, wherein amino acids at positions 1 and 2 are A and Q, the amino acid at position 256 is S and the amino acid at position 258 is G;
(xvi) a TCR beta chain-anti-CD3 having the sequence SEQ ID No: 16, wherein amino acids at positions 1 and 2 are A and Q, the amino acid at position 255 is S and the amino acid at position 258 is G;
(xvii) and a TCR beta chain-anti-CD3 having the sequence SEQ ID No: 16, wherein amino acid at positions 1 and 2 are A and I respectively, the amino acid at position 257 is S and the amino acid at position 258 is G;
(xviii) a TCR beta chain-anti-CD3 having the sequence SEQ ID No: 16, wherein amino acid at positions 1 and 2 are A and I respectively, the amino acid at position 256 is S and the amino acid at position 258 is G;
(xix) a TCR beta chain-anti-CD3 having the sequence SEQ ID No: 16, wherein amino acid at positions 1 and 2 are A and I respectively, the amino acid at position 255 is S and the amino acid at position 258 is G.

13. The TCR of claim 12, wherein the combination of alpha chain sequence and beta chain-anti-CD3 sequence is selected from:
the alpha chain amino acid sequence is (i) and the beta chain-anti-CD3 amino acid sequence is (vii);
the alpha chain amino acid sequence is (i) and the beta chain-anti-CD3 amino acid sequence is (x);
the alpha chain amino acid sequence is (vi) and the beta chain-anti-CD3 amino acid sequence is (ix);
the alpha chain amino acid sequence is (v) and the beta chain-anti-CD3 amino acid sequence is (viii);
the alpha chain amino acid sequence is (vi) and the beta chain-anti-CD3 amino acid sequence is (vii);
the alpha chain amino acid sequence is (i) and the beta chain-anti-CD3 amino acid sequence is (xi);
the alpha chain amino acid sequence is (i) and the beta chain-anti-CD3 amino acid sequence is (xii);
the alpha chain amino acid sequence is (i) and the beta chain-anti-CD3 amino acid sequence is (xiii);
the alpha chain amino acid sequence is (vi) and the beta chain-anti-CD3 amino acid sequence is (xiv);
the alpha chain amino acid sequence is (vi) and the beta chain-anti-CD3 amino acid sequence is (xv);
the alpha chain amino acid sequence is (vi) and the beta chain-anti-CD3 amino acid sequence is (xvi);
the alpha chain amino acid sequence is (v) and the beta chain-anti-CD3 amino acid sequence is (xvii);
the alpha chain amino acid sequence is (v) and the beta chain-anti-CD3 amino acid sequence is (xviii);
the alpha chain amino acid sequence is (v) and the beta chain-anti-CD3 amino acid sequence is (xix);
the alpha chain amino acid sequence is (vi) and the beta chain-anti-CD3 amino acid sequence is (xi);
the alpha chain amino acid sequence is (vi) and the beta chain-anti-CD3 amino acid sequence is (xii);
the alpha chain amino acid sequence is (vi) and the beta chain-anti-CD3 amino acid sequence is (xiii).

14. A nucleic acid encoding a TCR of claim 1.

15. A non-naturally occurring and/or purified and/or engineered cell, especially a T-cell, presenting a TCR as claimed in claim 1.

16. A cell harboring
(a) a TCR expression vector which comprises nucleic acid as claimed in claim 14 in a single open reading frame, or two distinct open reading frames encoding the alpha chain and the beta chain respectively; or
(b) a first expression vector which comprises nucleic acid encoding the alpha chain of a TCR as claimed in claim 1, and a second expression vector which comprises nucleic acid encoding the beta chain of a TCR as claimed in claim 1.

17. A pharmaceutical composition comprising a TCR as claimed in claim 1 or a cell as claimed in claim 15, together with one or more pharmaceutically acceptable carriers or excipients.

18. A TCR which binds the CLGGLLTMV peptide (SEQ ID NO: 1) (derived from the EBV LMP2A protein) presented as a peptide-HLA-A2 complex, or a cell expressing and/or presenting such a TCR, for use in medicine.


SEQUENCE LISTING (32 sequences)

SEQ ID NO: 1 - protein, 9 aa, Epstein Barr virus
CLGGLLTMV

SEQ ID NO: 2 - protein, 207 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSAEANSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 3 - protein, 242 aa, Homo sapiens
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLGGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 4 - protein, 207 aa, Artificial Sequence
  Description of Artificial Sequence Synthetic T cell receptor alpha chain extracellular domain
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSAEANSGYALNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 5 - protein, 242 aa, Artificial Sequence
  Description of Artificial Sequence Synthetic T cell receptor beta chain extracellular domain
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLGGYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 6 - protein, 207 aa, Artificial Sequence
  Description of Artificial Sequence Synthetic T cell receptor alpha chain variable domain
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSAEAHQPYRLNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 7 - protein, 207 aa, Artificial Sequence
  Description of Artificial Sequence Synthetic T cell receptor alpha chain variable domain
QKEVEQDPGPLSVPEGAIVSLNCTYSNSAFQYFMWYRQYSRKGPELLMYLPGGGNKEDGRFTAQVDKSSKYISLFIRDSQPSDSATYLCAMSADAYGHYALNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 8 - protein, 207 aa, Artificial Sequence
  Description of Artificial Sequence Synthetic T cell receptor alpha chain variable domain
QKEVEQDPGPLSVPEGAIVSLNCTYSNSAFQYFMWYRQYSRKGPELLMYQFQDGNKEDGRFTAQVDKSSKYISLFIRDSQPSDSATYLCAMSAEAHQPYRLNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 9 - protein, 207 aa, Artificial Sequence
  Description of Artificial Sequence Synthetic T cell receptor alpha chain variable domain
QKEVEQDPGPLSVPEGAIVSLNCTYSNPHMAYFMWYRQYSRKGPELLMYTYSSGNKEDGRFTAQVDKSSKYISLFIRDSQPSDSATYLCAMSADAYGHYALNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 10 - protein, 207 aa, Artificial Sequence
  Description of Artificial Sequence Synthetic T cell receptor alpha chain variable domain
QKEVEQDPGPLSVPEGAIVSLNCTYSNPHMAYFMWYRQYSRKGPELLMYTYSSGNKEDGRFTAQVDKSSKYISLFIRDSQPSDSATYLCAMSAEAHQPYRLNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 11 - protein, 207 aa, Artificial Sequence
  Description of Artificial Sequence Synthetic T cell receptor alpha chain variable domain
QKEVEQDPGPLSVPEGAIVSLNCTYSNPHMAYFMWYRQYSRKGPELLMYQFQDGNKEDGRFTAQVDKSSKYISLFIRDSQPSDSATYLCAMSADAYGHYALNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 12 - protein, 207 aa, Artificial Sequence
  Description of Artificial Sequence Synthetic T cell receptor alpha chain variable domain
QKEVEQDPGPLSVPEGAIVSLNCTYSNPHMAYFMWYRQYSRKGPELLMYQFQDGNKEDGRFTAQVDKSSKYISLFIRDSQPSDSATYLCAMSAEAHQPYRLNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 13 - protein, 242 aa, Artificial Sequence
  Description of Artificial Sequence Synthetic T cell receptor beta chain variable domain
EAGVAQSPRYKIIEKRQSVAFWCNPISGHATLYWYQQILGQGPKLLIQFVVAASVDDSQLPKDRFSAERLKGVDSTLKIQPAKLEDSAVYLCASSLGGYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 14 - DNA, 627 bp, Artificial Sequence
  Description of Artificial Sequence Synthetic T cell receptor alpha chain extracellular domain
atgcaaaaagaagttgaacaagatcctggaccactcagtgttccagagggagccatcgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatgagcgcggaagcaaattccgggtatgcactcaacttcggcaaaggcacctcgctgttggtcacaccccatatccagaaccctgaccctgccgtgtaccagctgagagactctaagtcgagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctaa

SEQ ID NO: 15 - DNA, 732 bp, Artificial Sequence
  Description of Artificial Sequence Synthetic T cell receptor beta chain extracellular domain
atggaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctctgtgccagcagccttggcgggtacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaa

SEQ ID NO: 16 - protein, 500 aa, Artificial Sequence
  Description of Artificial Sequence Synthetic T cell receptor anti-CD3 fusion protein
XXQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSEAGVAQSPRYKIIEKRQSVAFWCNPISGHATLYWYQQILGQGPKLLIQFVVAASVDDSQLPKDRFSAERLKGVDSTLKIQPAKLEDSAVYLCASSLGGYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 17 - protein, 9 aa, Epstein Barr virus
SLGGLLTMV

SEQ ID NO: 18 - protein, 9 aa, Epstein Barr virus
CLGGLITMV

SEQ ID NO: 19 - DNA, 45 bp, Artificial Sequence
  Description of Artificial Sequence Synthetic primer
gaattccatatgcaaaaagaagttgaacaagatcctggaccactc

SEQ ID NO: 20 - DNA, 36 bp, Artificial Sequence
  Description of Artificial Sequence Synthetic primer
ttgtcagtcgacttagagtctctcagctggtacacg

SEQ ID NO: 21 - DNA, 45 bp, Artificial Sequence
  Description of Artificial Sequence Synthetic primer
gaattccatatggaagctggagttgctcaatctcccagatataag

SEQ ID NO: 22 - DNA, 32 bp, Artificial Sequence
  Description of Artificial Sequence Synthetic primer
tagaaaccggtggccaggcacaccagtgtggc

SEQ ID NO: 23 - protein, 5 aa, Artificial Sequence
  Description of Artificial Sequence Synthetic linker sequence
GGGGS

SEQ ID NO: 24 - protein, 5 aa, Artificial Sequence
  Description of Artificial Sequence Synthetic linker sequence
GGGSG

SEQ ID NO: 25 - protein, 5 aa, Artificial Sequence
  Description of Artificial Sequence Synthetic linker sequence
GGSGG

SEQ ID NO: 26 - protein, 5 aa, Artificial Sequence
  Description of Artificial Sequence Synthetic linker sequence
GSGGG

SEQ ID NO: 27 - protein, 6 aa, Artificial Sequence
  Description of Artificial Sequence Synthetic linker sequence
GSGGGP

SEQ ID NO: 28 - protein, 5 aa, Artificial Sequence
  Description of Artificial Sequence Synthetic linker sequence
GGEPS

SEQ ID NO: 29 - protein, 7 aa, Artificial Sequence
  Description of Artificial Sequence Synthetic linker sequence
GGEGGGP

SEQ ID NO: 30 - protein, 12 aa, Artificial Sequence
  Description of Artificial Sequence Synthetic linker sequence
GGEGGGSEGGGS

SEQ ID NO: 31 - protein, 29 aa, Artificial Sequence
  Description of Artificial Sequence Synthetic peptide
RTSGPGDGGKGGPGKGPGGEGTKGTGPGG

SEQ ID NO: 32 - protein, 9 aa, Artificial Sequence
  Description of Artificial Sequence Synthetic peptide
EVDPIGHLY